SEQUENCE LISTING (38 sequences)

SEQ ID NO: 1 - DNA, 20 bp, Artificial Sequence
  Chemically synthesized
ccagagcatctacgccaagt

SEQ ID NO: 2 - DNA, 20 bp, Artificial Sequence
  Chemically synthesized
cacgtcgtagagggagaagg

SEQ ID NO: 3 - DNA, 20 bp, Artificial Sequence
  Chemically synthesized
gcagaggtcctatcccatga

SEQ ID NO: 4 - DNA, 22 bp, Artificial Sequence
  Chemically synthesized
ctgggtggaaagagagaagctg

SEQ ID NO: 5 - DNA, 24 bp, Artificial Sequence
  Chemically synthesized
attcaacgaagcccactacccaga

SEQ ID NO: 6 - DNA, 24 bp, Artificial Sequence
  Chemically synthesized
atccttggctgacttgaggatgga

SEQ ID NO: 7 - DNA, 20 bp, Artificial Sequence
  Chemically synthesized
tcaccagggctgcttttaac

SEQ ID NO: 8 - DNA, 20 bp, Artificial Sequence
  Chemically synthesized
gcaaagcttcccgttctcag

SEQ ID NO: 9 - DNA, 22 bp, Artificial Sequence
  Chemically synthesized
tagcatctactgcaaggaagac

SEQ ID NO: 10 - DNA, 20 bp, Artificial Sequence
  Chemically synthesized
gtgataaaccaagtcccgag

SEQ ID NO: 11 - DNA, 20 bp, Artificial Sequence
  Chemically synthesized
caaaggcaaacaacccactt

SEQ ID NO: 12 - DNA, 20 bp, Artificial Sequence
  Chemically synthesized
tctgctggaggctgaggtat

SEQ ID NO: 13 - DNA, 24 bp, Artificial Sequence
  Chemically synthesized
cgagcaatttgccaagctcctgaa

SEQ ID NO: 14 - DNA, 24 bp, Artificial Sequence
  Chemically synthesized
ttcgggcactgcaggaacaaattc

SEQ ID NO: 15 - DNA, 20 bp, Artificial Sequence
  Chemically synthesized
tctaatcgaagggccaaatg

SEQ ID NO: 16 - DNA, 20 bp, Artificial Sequence
  Chemically synthesized
tgtgagggctgtgtctgttc

SEQ ID NO: 17 - DNA, 20 bp, Artificial Sequence
  Chemically synthesized
gaatctcgaaatctcagccc

SEQ ID NO: 18 - DNA, 21 bp, Artificial Sequence
  Chemically synthesized
cttcactaatttgctcaggac

SEQ ID NO: 19 - DNA, 21 bp, Artificial Sequence
  Chemically synthesized
ggaatgtgatgtatgatagcc

SEQ ID NO: 20 - DNA, 19 bp, Artificial Sequence
  Chemically synthesized
tgatttcggtttgttctgg

SEQ ID NO: 21 - DNA, 28 bp, Artificial Sequence
  Chemically synthesized
ggactagttgaatgtacagcatgatgga

SEQ ID NO: 22 - DNA, 29 bp, Artificial Sequence
  Chemically synthesized
cggaattcgatgtgtgtcagtggcatggt

SEQ ID NO: 23 - DNA, 20 bp, Artificial Sequence
  Chemically synthesized
gcagaggtcctatcccatga

SEQ ID NO: 24 - DNA, 22 bp, Artificial Sequence
  Chemically synthesized
ctgggtggaaagagagaagctg

SEQ ID NO: 25 - DNA, 20 bp, Artificial Sequence
  Chemically synthesized
tcaccagggctgcttttaac

SEQ ID NO: 26 - DNA, 20 bp, Artificial Sequence
  Chemically synthesized
gcaaagcttcccgttctcag

SEQ ID NO: 27 - DNA, 22 bp, Artificial Sequence
  Chemically synthesized
tagcatctactgcaaggaagac

SEQ ID NO: 28 - DNA, 20 bp, Artificial Sequence
  Chemically synthesized
gtgataaaccaagtcccgag

SEQ ID NO: 29 - DNA, 20 bp, Artificial Sequence
  Chemically synthesized
caaaggcaaacaacccactt

SEQ ID NO: 30 - DNA, 20 bp, Artificial Sequence
  Chemically synthesized
tctgctggaggctgaggtat

SEQ ID NO: 31 - DNA, 24 bp, Artificial Sequence
  Chemically synthesized
cgagcaatttgccaagctcctgaa

SEQ ID NO: 32 - DNA, 24 bp, Artificial Sequence
  Chemically synthesized
ttcgggcactgcaggaacaaattc

SEQ ID NO: 33 - DNA, 20 bp, Artificial Sequence
  Chemically synthesized
tctaatcgaagggccaaatg

SEQ ID NO: 34 - DNA, 20 bp, Artificial Sequence
  Chemically synthesized
tgtgagggctgtgtctgttc

SEQ ID NO: 35 - DNA, 20 bp, Artificial Sequence
  Chemically synthesized
gaatctcgaaatctcagccc

SEQ ID NO: 36 - DNA, 21 bp, Artificial Sequence
  Chemically synthesized
cttcactaatttgctcaggac

SEQ ID NO: 37 - DNA, 21 bp, Artificial Sequence
  Chemically synthesized
ggaatgtgatgtatgatagcc

SEQ ID NO: 38 - DNA, 19 bp, Artificial Sequence
  Chemically synthesized
tgatttcggtttgttctgg

(19) United States
(12) Patent Application Publication
(10) Pub. No.: US 2022/0364049 A1
(43) Pub. Date: Nov. 17, 2022

(54) COMPOSITION AND METHODS FOR CULTURING RETINAL PROGENITOR CELLS

(71) Applicant: Yale University, New Haven, CT (US)

(72) Inventors: Lawrence Rizzolo, Guilford, CT (US); Deepti Singh, East Haven, CT (US)

(21) Appl. No.: 17/845,461

(22) Filed: Jun. 21, 2022

Related U.S. Application Data
(63) Continuation of application No. 16/177,728, filed on Nov. 1, 2018, now abandoned.
(60) Provisional application No. 62/580,356, filed on Nov. 1, 2017.

(51) Int. Cl. C12N 5/00; C12N 5/079; C12N 5/074; C12N 5/0735; A61L 27/38; A61L 27/54; A61L 27/56; A61L 27/50; A61L 27/26

(52) U.S. Cl. CPC C12N 5/0062; C12N 5/0621; C12N 5/0696; C12N 5/0606; A61L 27/3886; A61L 27/54; A61L 27/56; A61L 27/50; A61L 27/3891; A61L 27/26; A61L 27/3834; C12N 2533/52; C12N 2533/54; C12N 2533/80; C12N 2513/00; A61L 27/3604

(57) ABSTRACT

The present invention provides a scaffold for culturing retinal tissue comprising an amount of gelatin, an amount of chondroitin sulfate, an amount of hyaluronic acid, wherein the amount of gelatin, chondroitin sulfate, and hyaluronic acid are prepared into a three-dimensional monolith, wherein the monolith is sectioned into planar sheets, and an amount of laminin-521.

### CROSS REFERENCE TO RELATED APPLICATIONS

This application is a continuation of U.S. patent application Ser. No. 16/177,728, filed Nov. 1, 2018, which claims priority to U.S. Provisional Application No. 62/580,356, filed Nov. 1, 2017, each of which is hereby incorporated by reference herein in its entirety.

### STATEMENT REGARDING FEDERALLY SPONSORED RESEARCH OR DEVELOPMENT

This invention was made with government support under W81XWH-15-1-0029, awarded by the United States Department of Defense. The government has certain rights in the invention.

### REFERENCE TO A “SEQUENCE LISTING,” A TABLE, OR A COMPUTER PROGRAM LISTING APPENDIX SUBMITTED AS AN ASCII TEXT FILE

The Sequence Listing written in the ASCII text file:

“047162_5246_01US SequenceListing.txt”; created on Jun. 21, 2022, and 7,740 bytes in size, is hereby incorporated by reference.

### BACKGROUND OF THE INVENTION

Retinal degeneration and its associated loss of photoreceptors is a major cause of blindness that affects millions worldwide. Two major disorders are age-related macular degeneration (AMD) and retinitis pigmentosa (RP) (Wert K J, et al., Dev. Ophthalmol., 2014, 53:33-43). One approach to treat these diseases is to inject photoreceptor precursors, derived from stem cells or isolated from developing mouse retina, into the subretinal space. Encouraging results have been obtained, but the efficiency is low (reviewed in Reh T A, Invest. Ophthalmol. Vis. Sci., 2016, 57:ORSFg1-7). Further, there is some question whether transplanted cells integrated or transferred cytoplasm to host cells (Pearson R A, et al. Nat. Commun., 2016, 7:13029; Santos-Ferreira T, et al., Nat. Commun., 2016, 7:13028).

Stem cells are readily differentiated into retinal precursor cells (RPC) using two types of spherical retinoids that mimic retinogenesis (Ohlemacher S K, et al., Curr. Protoc. Stem Cell Biol. John Wiley & Sons, Inc., 2007:pp 1H.8.1-20; Meyer J S, et al. Proc. Natl. Acad. Sci. USA, 2009, 106:16698-16703; Eiraku M, et al. Nature, 2011, 472:51-56; Meyer J S, et al. Stem Cells, 2011, 29:1206-1218; Nakano T, et al. Cell Stem Cell, 2012, 10:771-785; Reichman S, et al., Proc. Natl. Acad. Sci. U.S.A., 2014, 111:8518-8523; Zhong X, et al., Nat. Commun., 2014, 5:4047; Mellough CB, et al. Stem Cells, 2015, 33:2416-2430; Kaewkhaw R, et al. Invest. Ophthalmol. Vis. Sci., 2016, 57:ORSFl 1-11). The retinoids are laminated and have been used to study mechanisms of retinal disease by producing them from patient-derived induced pluripotent cells (Tucker BA, et al., Elife, 2013, 2:e00824; Burnight E R, et al. Gene Ther., 2014, 21:662-672; Arno G, et al., Am. J. Hum. Genet., 2016, 99:1305-1315.; Ohlemacher S K, et al., Stem Cells, 2016, 34:1553-1562; Parfitt D A, et al., Cell Stem Cell, 2016, 18:769-781). Nonetheless, the spherical retinoid model has significant limitations due to its geometry. The outer surface of the spheroid is the photoreceptor layer, but photoreceptors are sparse and ill formed, likely because they are not in direct contact with a layer of retinal pigment epithelium (RPE). The inner surface contains ganglion cells, but these cells begin to die as photoreceptors begin to mature (Ohlemacher S K, et al., Stem Cells, 2016, 34:1553-1562). Further the small lumen of the retinoid makes it difficult to model the effects of adding a potential therapeutic agent into the vitreous. For tissue replacement therapy, the retinoids are unable to integrate simultaneously with the host neurosensory retina and RPE (Assawachananont J, et al. Stem Cell Reports, 2014, 2:662-674; Shirai H, et al., Proc. Natl. Acad. Sci. U.S.A., 2016, 113:E81-E90).

Scaffolds have been used in attempts to culture a planar retina. Different materials used for fabricating scaffold for retinal regeneration include polycaprolactone (PCL), poly(DL-lactic-co-glycolic acid, poly(glycol) acid and poly (lactic) acid (Giordano, G., et al., Biomed. Mater. Res., 1997, 34:87-93; Lavik E B, et al. Biomaterials, 2005, 26:3187-3196; Engler A J, et al, Cell, 2006, 126:677-689; Tao S, et al., Lab on a Chip, 2007, 7:695-701; Redenti S, et al., J. Ocul. Biol. Dis. Infor., 2008, 1:19-29; Gilbert P M, et al., Science, 2010, 329:1078-1081; Hynes, S R Lavik, E B, Graefes Arch. Clin. Exp. Ophthalmol., 2010, 248:763-778; Kador K E, Goldberg J L, Expert Rev. Ophthalmol., 2012, 7:459-470; Chen, H L, Int. J. Nanomedicine, 2011, 6:453-461). Natural and artificial substrates have been found to affect cell behavior (Aizawa Y, Shoichet M S, Biomaterials, 2012, 33:5198-5205; Nasu M, et al. PLoS ONE, 2012, 7:e53024; Reinhard J, et al., Exp. Eye Res., 2015, 133:132-140; Steedman, M R, et al., Biomed. Microdevices, 2010, 12:363-369; Worthington, Kans., et al., Biomacromolecules, 2016,17:1684-1695). Scaffolds provide a niche for cells to proliferate and differentiate and can favor the differentiation of RPCs into photoreceptor precursors (Tomita M, et al., Stem Cells, 2005, 23:1579-1588; Yao J, et al., Tissue Eng. Part A, 2015, 21:1247-1260). Scaffolds are also able to absorb the pressures of implantation procedures, while keeping the cells intact and stress free (Ballios B G, et al, Biomaterials, 2010, 31:2555-2564; Kraehenbuehl T P, et al., Nat. Meth., 2011, 8:731-736). Although these scaffolds might be suitable for implanting RPC into the subretinal space, they do not support differentiation into laminated retinoids, as do the spherical retinoids. Therefore, they cannot be used to study retinal differentiation.

Thus, there is a need for suitable scaffolds for generating laminated retinoids for implantation, and for properly studying retinal differentiation, patient-specific mechanisms of retinal disease, and the mechanism of action for putative therapeutic agents. The present invention meets this unmet need.

### SUMMARY OF THE INVENTION

The present invention provides a scaffold for culturing retinal tissue that includes laminin, a three-dimensional monolith that is sectioned into planar sheets and comprises gelatin, chondroitin sulfate, and hyaluronic acid. In some embodiments, the laminin is laminin-521. In some embodiments, the three-dimensional monolith is formed by crosslinking. In some embodiments, the three-dimensional monolith is frozen and lyophilized. In some embodiments, the scaffold is seeded with cells such as retinal progenitor cells. In some embodiments, the retinal progenitor cells are derived from human embryonic stem cells. In some embodiments, the retinal progenitor cells are derived from human inducible pluripotent stem cells.

In some embodiments, the scaffold of the present invention is seeded on top of a monolayer of cells, wherein the monolayer of cells are retinal pigment epithelial cells. In some embodiments, the retinal pigment epithelial cells are human fetal retinal pigment epithelial cells. In some embodiments, the retinal pigment epithelial cells are derived from stem cells such as human embryonic stem cells and human inducible pluripotent stem cells.

In some embodiments, the monolith of the present invention comprises a ratio of concentrations of gelatin, chondroitin sulfate, and hyaluronic acid wherein the ratio is 2:1:2.

In some embodiments, the three-dimensional monolith is sectioned into planar sheets the planar sheets comprise a thickness of about 60 μm.

In some embodiments, the present invention further provides a retinal coculture system for generating retinal implants that includes a planar scaffold constructed of gelatin, chondroitin sulfate, and hyaluronic acid; a monolayer of differentiated retinal pigment epithelial cells; and a population of retinal progenitor cells. The planar scaffold is seeded with cells from the population of retinal progenitor cells, is placed on top of the monolayer of differentiated retinal pigment epithelial cells and is incubated with media. In some embodiments, the retinal coculture system comprises laminin-521. In some embodiments, the retinal pigment epithelial cells are human fetal retinal pigment epithelial cells. In some embodiments, the retinal progenitor cells are derived from human embryonic stem cells. In some embodiments, the retinal progenitor cells are derived from human inducible pluripotent stem cells.

In some embodiments, the present invention provides a method of generating retinal implants that includes the following steps: a) generating a scaffold for culturing retinal tissue comprising an amount of gelatin, an amount of chondroitin sulfate, an amount of hyaluronic acid, wherein the amount of gelatin, chondroitin sulfate, and hyaluronic acid are prepared into a three-dimensional monolith, wherein the monolith is sectioned into planar sheets, and an amount of laminin-521; b) seeding the scaffold with retinal progenitor cells; c) placing the seeded scaffold in direct contact with a monolayer of retinal pigment epithelial cells; thereby creating a coculture assembly; d) incubating the coculture assembly thereby generating an organoid; and e) implanting the generated organoid into the subretinal space of a subject. In some embodiments, the retinal progenitor cells are human embryonic stem cells. In some embodiments, the retinal pigment epithelial cells are human fetal retinal pigment epithelial cells.

### BRIEF DESCRIPTION OF THE DRAWINGS

The foregoing summary, as well as the following detailed description of embodiments of the invention, will be better understood when read in conjunction with the appended drawings. It should be understood, however, that the invention is not limited to the precise arrangements and instrumentalities of the embodiments shown in the drawings. In the drawings:

FIG. 1 depicts a schematic of the co-development of RPE and retina. RPC are plated on a microporous scaffold and layered on top of a monolayer of RPE. Each tissue modulates the differentiation of the other. Simultaneously, intrinsic signaling among differentiating RPC may also regulate development. As depicted, hESCs or hiPSCs form immature spherical organoids (RPC), are dissociated and reseeded on porous scaffolds, and co-cultured alone or with RPE monolayers. In the panel on the top, the thick arrows suggest RPC and RPE co-differentiate by sending signals back and forth throughout development by establishing gradients of tropic factors. In the panel on the bottom, the thick arrows indicate RPC also differentiates via intrinsic factors RPE-RPC signaling and intrinsic RPC signaling are simultaneous. Development can be perturbed by using a disease model for RPE.

FIG. 2 depicts the instrumentation for the compressive strength testing of the scaffolds.

FIG. 3A through FIG. 3F depict embryoid bodies invading the porous structure of the gelatin/chondroitin/hyaluronic acid (GCH) scaffold. FIG. 3A is a scanning electron micrograph showing three faces of a block of scaffold. FIG. 3B shows higher magnification of one face. Light micrograph (FIG. 3C) and scanning electron micrograph (FIG. 3D) show embryoid bodies on the scaffold one-day post-seeding. FIG. 3E depicts cultures that were stained with DAPI (blue) one-week post-seeding to reveal cell nuclei. Three-dimensional reconstruction from confocal micrographs demonstrated that cells migrated the thickness of the scaffold. FIG. 3F illustrates that after three weeks, cells homogenously populated the entire scaffold. Representative image of 3rd week of embryoid bodies on GCH (FIG. 3F). Scale Bar: FIG. 3A, 1000 μm; FIG. 3B and FIG. 3C, 500 μm; FIG. 3D, 100 μm; FIG. 3E and FIG. 3F, scale in μm.

FIG. 4A through FIG. 4F present results showing that the GCH scaffold enhanced the differentiation of RPC from WA09 embryoid bodies. FIG. 4A demonstrates that the percentage of proliferating (Ki67-positive) cells decreased with time. This decrease occurred earlier in the GCH cultures. FIG. 4B demonstrates that caspases were not expressed and poly (ADP-ribose) polymerase-1 (PARP-1)-cleavage products were undetected indicating that apoptosis did not account for the decrease in proliferation. FIG. 4C demonstrates that, as assayed by qRT2-PCR, the mRNA for the early eye field gene, LHX2, increased, and pluripotency markers, OCT4 and Nanog, decreased. Sox1, an anterior forebrain marker, was unchanged. As shown in FIG. 4D, on D24, the expression of early eye field genes on the scaffold was compared to suspension cultures. Equal expression in both cultures=1. As shown in FIG. 4E, after the fifth week, the cultures were assayed using qRT2-PCR (see FIG. 5B for a map of the array and its controls). WA09 cells in suspension were compared with WA09 cultured on the scaffold. The mRNAs plotted above the red line were expressed on the scaffold>4×the expression in cell suspension. The mRNAs plotted below the red line were expressed on the scaffold<4×the expression in cell suspension. As shown in FIG. 4F, protein expression was monitored by immunoblotting. Rhodopsin was detected as early as D51.

FIG. 5A through FIG. 5C present results showing that the GCH scaffold also enhances the differentiation of RPC from induced pluripotent stem cell (iPSC) embryoid bodies. A human iPSC line, Y6, differentiated on the GCH scaffold. Embryoid bodies were differentiated and seeded on scaffolds, as described for WA09 cells in Methods and seeded on the scaffold on D21. FIG. 5A demonstrates that after 4 weeks of differentiation on the scaffold (D67), PCR products for retinal genes could be identified by acrylamide gel electrophoresis. FIG. 5B depicts the microarray used for qRT2-PCR comparisons between D67 versus DO cultures and D67 WA09 vs Y6 cultures on the GCH scaffold, along with the results. GAPDH and actin (ACT) were used to normalize the data. The data were color coded according to the map of the array shown at the bottom. Y6 differentiated substantially by D67, and only minor differences were observed between WA09 and Y6 cells on the GCH scaffold. Red line, 4×over expression; Green line, 4×under-expression. FIG. 5C presents confocal immunofluorescence micrographs that show 1) evidence of proliferating cells on D21, when cells were seeded on the scaffold (Ki-67), 2) early eye field markers (OTX2 and VSX2) were evident on D28 and D31, and 3) the photoreceptor marker, RCVRN was evident on D65.

FIG. 6 depicts results from experiments using confocal immunocytochemistry to confirm differentiation on the scaffold. Cells were seeded on the scaffold on D7. Cell nuclei were counterstained with DAPI (blue). Immunoreactivity for Ku80 antigen confirmed the cultures were derived from human cells. PAX6 and RAX, master regulators of retinal differentiation were evident by D14. Sox1, an anterior forebrain marker was still evident. By D28 neural retinal markers CRX, CHX10, and OTX2 were evident. By D38, the photoreceptor marker, recoverin (RCVRN) appeared along with ganglion cell marker, HuC/D. By D67, SOX9 (Muller Glia), rhodopsin (Rho, rods), and BRN3 (ganglion cells) were evident. MITF immunoreactivity indicated that RPE was also present. Asterisk; auto fluorescence of the scaffold. Scale Bar, 20 μm

FIG. 7 depicts results from experiments using confocal immunocytochemistry to confirm that on D31, early markers for retinal differentiation co-localized in many cells. Cells in each row were double labeled and the images from each antibody channel were false-colored red or green, which made identical cells in the merged images (without DAPI) appear yellow/orange. Most labeled cells were co-labeled with two RPC markers. All PAX6 cells appeared to co-label with VSX2, but the reverse was not true. There was extensive, but incomplete overlap between VSX2 and RAX. Scale Bar, 20 μm

FIG. 8 depicts results from experiments using confocal immunocytochemistry to confirm that on D90, early markers for retinal differentiation co-localized in many cells. Cells in each row were double labeled and the images from each antibody channel were false-colored red or green, which made identical cells in the merged images (without DAPI) appear yellow/orange. Antibodies were used to identify retinal ganglion cells (BRN3, HuC/D), photoreceptor precursors (CRX, RCVRN), bipolar cells (VSX2), and RPE (MITF). No overlap was observed, indicating that at this stage distinct cell types had emerged, but were often intermixed. In other words, cells failed to segregate into distinct lamina. Note the orange signal does not indicated co-localization of BRN3 and RCVRN, because BRN3 should be concentrated in the nucleus, but RCVRN should be concentrated in the cytoplasm. Arrowheads, nucleus of a RCVRN-positive cell; Arrows, nucleus of a BRN3-positive cell. Scale Bar, 20 μm

FIG. 9 depicts results from experiments demonstrating that the GCH scaffold failed to elicit and immune response. The scaffold was implanted into the sub-retinal space of rd10 mice on P30 and the eyes harvested 3 weeks later (P51) for sectioning and immunofluorescence staining. By this time, the scaffold had been resorbed and retinal vessels were used to map the location of the implant. The tissue sections were counterstained for recoverin (RCVRN) to reveal the location of photoreceptors and DAPI to reveal the nuclear layers. Upper panels: Because the antibody for IL-6 was a mouse monoclonal (green), background fluorescence was observed in the vascular bed in the inner nuclear layers (arrowhead). IL-6 was slightly increased in the choroid relative to the un-operated control retina (CTL); an IL-6 positive cell is indicated by the short arrows. Minimal immunofluorescence was observed near the subretinal space (long arrows). Lower panels: Microglia marker, IBA-1, immunoreactivity was slight in the outer nuclear layer near the implant site (subretinal space). There was no clear increase in the presence of IBA-1 positive cells in comparison with the control retina. DIC, differential interference contrast; Retinal layers: a, ganglion cells; b, inner plexiform layer: c, inner nuclear layer; d, outer plexiform layer; e, ONL; f, RPE; g, choroid. Note that in the mouse the RPE and choroid are both highly pigmented and might quench a fluorescent signal. Scale Bar, 20 μm

FIG. 10 depicts results from experiments demonstrating that the WA09-GCH tissue graft survived at least 12 weeks in the sub-retinal space of rd10 mice. Micrographs were acquired with a 40×oil objective, except the right column, which was acquired with a 100×oil objective. The red boxes indicate the regions acquired at higher magnification. The grafts (D21) were implanted at P30, when the ONL was >75% degenerated. Globes were harvested for sectioning on the days indicated at the left. By P44, the ONL was reduced to one, discontinuous row of cells in the un-operated control retina (CTR). TRA-1-85, an antibody to a membrane antigen was observed alone in the position of microvilli (white arrowhead) and with recoverin in the body of the cells (long arrow). Three to four rows of recoverin-positive were observed in the ONL, which likely included single and double-labeled cells. Six to eight-weeks post implantation (P72, P84), at least one continuous row of cells was observed in the ONL that was positive for human antigen and recoverin. Ku-80 labels the double stranded DNA of human nuclei and mitochondria. Therefore, it can distinguish between cell implantation (nucleus and cytoplasm labeled) and cytoplasm transfer (only cytoplasm labeled). Because cytoplasm, but not nuclei, labeled in the ONL, cytoplasm was transferred between the implant and the host (short arrows). Note that recoverin is green in this row of images. The processes of an inner neuron are revealed by clustered mitochondria along the neuronal processes (blue arrowheads). The maximum intensity projection (MIP) image traces the arborization of the cell marked by the arrow. The merged images of multiple confocal images were collapsed into one image. The arbor extends laterally in the inner plexiform layer and towards the ONL. Because the nucleus of this cell was also labeled, this is an example of cell implantation rather than cytoplasmic transfer. DIC, differential interference contrast; Retinal layers: a, ganglion cells; b, inner plexiform layer: c, inner nuclear layer; d, outer plexiform layer; e, ONL; f, RPE; g, choroid. Asterisk, retinal detachment; Scale Bar, 20 μm; 100×Bar, 5 μm

FIG. 11 depicts results from experiments demonstrating that 6 weeks post-implantation (P72), control procedures have minimal effect on retinal degeneration. For reference, the unoperated eye exhibits one discontinuous row of recoverin-positive cells (short arrows). TRA-1-85 non-specific staining was observed in the choroid (arrowheads). Injection of a suspension of cells resulted in clumps of cells in the sub-retinal space that were positive for TRA-1-85, but not recoverin (long arrows). Only one discontinuous row of recoverin-positive, TRA-1-85-negative cells was observed. Similarly, implantation of just the scaffold had minimal effect. Retinal layers: a, ganglion cells; b, inner plexiform layer: c, inner nuclear layer; d, outer plexiform layer; e, 5 ONL; f, RPE; g, choroid. Scale Bar, 20 μm

FIG. 12 depicts results from experiments demonstrating that retinal cups derived from human embryonic stem cells were partially dissociated and fully populated a laminin-521 coated GCH scaffold. After 1 day, cells (blue) adhered to the scaffold (blue-green autofluorescence. After 4 weeks, post seeding cells homogenously populated the scaffold, in contrast to uncoated GCH where cellular voids were observed (FIG. 3A-FIG. 3F). Day 7, enface view, Scale Bar, 200 μm; Day 7, 3-dimensional reconstruction, scale in microns; Day 7, 3-dimensional reconstruction, scale in microns.

FIG. 13A through FIG. 13D, depicts results from experiments demonstrating that laminin 521 promotes the differentiation of RPC. RPC were isolated on D25 and placed on GCH or GCH-521. Gene expression was assayed by qRT2-PCR. As shown in FIG. 13A and FIG. 13B, Stem cell markers Nanog (FIG. 13A) and OCT-4 (FIG. 13B) were down-regulated relative to DO more rapidly in GCH-521 cultures. FIG. 13C depicts that one week after plating, expression of mRNA increased for GCH-521 cultures relative to GCH cultures for the early eye field genel, PAX6, SIX3 and SIX6, along with recoverin (RCVRN), a photoreceptor marker. FIG. 13D depicts that by D67, there were additional changes in gene expression relative to the GCH cultures. Expression of the mRNA for pluripotency markers, OCT4 and Nanog, was substantially lower. Expression was upregulated for PAX6, CHX10, SIX3, SIX6, and NeuroD1. Equal expression in both cultures=1.

FIG. 14 illustrates results from confocal immunocytochemistry experiments confirming differentiation on the GCH-521 scaffold. Retinal cups were seeded on the scaffold on D21. Cell nuclei were counterstained with DAPI (blue). CHX10 a critical transcription factor for photoreceptor differentiation was found in 2 weeks post culturing of retinal cups on GCH-521 scaffold. Along with PAX6, a master regulator of retinal differentiation and LHX2 an important early field transcription factor. Scale Bar, 20 μm.

FIG. 15 depicts results from experiments demonstrating that five weeks post differentiation neural retinal markers CRX, OTX2 and recoverin was evident in GCH-521 culture. CRX and recoverin could be seen concentrating at the outer edge of the scaffold where as OTX2 positive cells were more homogenously distributed within the scaffold. Scale Bar, 20 μm.

FIG. 16 depicts results from experiments demonstrating that on the sixth week, the more matured marker HuC/D which is a ganglion cell marker was evident near the scaffold, and a marker for rod specific cells calretinin was evident near the free edge (away from the scaffold). Ku80 staining was performed to confirm the human origin of cells. Scale Bar, 20 μm.

FIG. 17 depicts results from experiments demonstrating that by D77, segregation of cell types is clearly evident in GHC-521 cultures. On GCH-521 scaffolds, ganglion cell markers, Brn3 and HuC/D, were found in and about the scaffold, but the photoreceptor marker, rhodopsin (Rho), was found away from the scaffold along the free edge of the neo-tissue. In the bottom row, a lower magnification shows recoverin (Rcvrn) and Brn3 separated by an unlabeled layer of cells. Asterisk; auto fluorescence scaffold. Scale Bar, 20 μm.

FIG. 18 depicts results for experiments demonstrating that after 8 months of culture photoreceptor and ganglion-like cells are still in evidence. A confocal plane acquired near the free surface (red arrowheads on the XZ and YZ planes) show a reticular network of fibers and cell bodies that are labeled by the photoreceptor marker, recoverin (red). A confocal plane acquired near the scaffold (green arrowheads on the XZ and YZ planes), show cell nuclei labeled by DAPI (blue) and BRN3 (green). The same XZ plane is shown twice, with and without DAPI. Long arrows, recoverin-positive cells; Short arrow, BRN3-positive nuclei; Scale bar, 20 mm

FIG. 19A and FIG. 19B show the results of labeling 8-month cultures with red-green opsin (red). FIG. 19A demonstrates a cone-shaped cell with a long process in three planes of section. The arrow indicates the same position in each plane. FIG. 19B demonstrates the three-dimensional reconstruction of a second cone-shaped cell. The arrows indicate a long outer segment-like structure filled with red-green opsin label. The asterisk indicates the scaffold. Processes as long as 70 mm were observed. Samples were counter-stained with DAPI to reveal nuclei. N, nucleus; asterisks, scaffold; Scale bar, 10 mm.

FIG. 20A through FIG. 20D demonstrate results from experiments demonstrating that the co-culture of RPE and RPC affects the differentiation of each neo-tissue. FIG. 20A depicts that cells remain viable and metabolically active in co-culture. FIG. 20B illustrates that the WA09 cells have little electrical resistance. The TER of RPE is significantly higher in co-culture than in mono-culture, p<0.01. FIG. 20C and FIG. 20D illustrates that the mRNAs represented by black dots lie along a 45° line, because their expression is not significantly changed. The red line indicates a 4×increase in gene expression; the green line indicates a 4×decrease in gene expression. The expression of RPE signature genes increased (red dots) due to co-culture. FIG. 20D demonstrates that the expression of photoreceptor and ganglion cell genes increased (Red dots), and the expression of interneurons decreased (green dots). Muller glia markers both increased and decreased.

FIG. 21A and FIG. 21B illustrate the method for measuring the TER of the co-culture and demonstrates the effects of culture. FIG. 21A illustrates how the culture is suspended in a culture dish to separate the dish into two chambers that emulate the vitreous and choroid. Electrodes can be placed without breaking sterility in each chamber and measure the resistance to an electrical current across the tissue (transepithelial electrical resistance, TER). The retinal organoid can be placed on a monolayer of RPE (red), as illustrated or on the bare filter (blue), which offers minimal resistance. After the experiment, mRNA or protein can be isolated for analysis, or the culture can be prepared for electron microscopy or immunofluorescence. FIG. 21B demonstrates the retinal organoid alone offers no resistance, but co-culture increases the resistance of RPE, regardless of whether the retinal organoid is derived from human induced pluripotent cells (Y6) or human embryonic stem cells (H9 aka WA09).

FIG. 22A through FIG. 22C depict results from experiments demonstrating that after 90 days of co-culture, a thicken layer of recoverin+ photoreceptor precursors are evident due to RPE, but RPE by itself does not provide polarity cues. FIG. 22A is a row of images depicting a short arrow highlighting colocalization of recoverin and Lhx2 and a long arrow highlighting Lhx2 alone (destined to become muller glia). Neither lie close to the scaffold. FIG. 22B is a row of images and depicts an enlarged image of the recoverin+ layer. FIG. 22C is a row of images and depicts that on the GCH scaffold without laminin 521, a thickened layer of recoverin+ cells could be found near and within the scaffold. *scaffold; Scale Bar, FIG. 22A: 50 μm; Scale Bars, FIG. 22B and FIG. 22C: 20 μm.

FIG. 23A through FIG. 23C illustrate results from experiments demonstrating the appearance of implanted cells 3 weeks post implantation (P51).

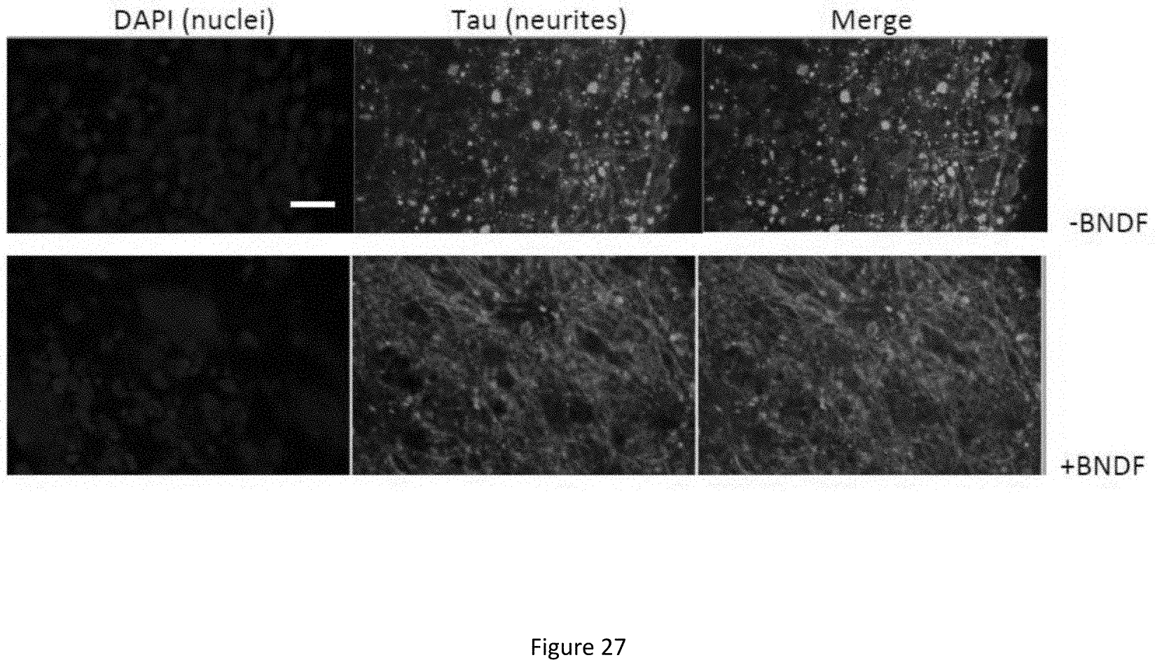

Remnants of the scaffold are autofluorescent and especially bright in the red channel (asterisk). The scaffold was over-exposed in FIG. 23A and FIG. 23B to reveal the TRA-1-85 signal (human antigen) in the implanted cells. The ONL and implanted cells were revealed by recoverin and cell nuclei by DAPI. In FIG. 23A, the right arrow points to the transition from multilayered recoverin-labelled cells in the ONL to the discontinuous monolayer of host ONL that is typical of this age. The region between the arrows is enlarged in FIG. 23B. In FIG. 23B, multilayered ONL lies between the arrowheads. Tra-1-85 is most intense in the region of photoreceptor outer/inner segments. In FIG. 23C the box in FIG. 23A is enlarged to reveal implanted cells in and about the scaffold. Arrowheads indicate the same location in each image. Scale Bar FIG. 23A, 50 μm; Scale Bar FIG. 23B and FIG. 23C, 20 μm.

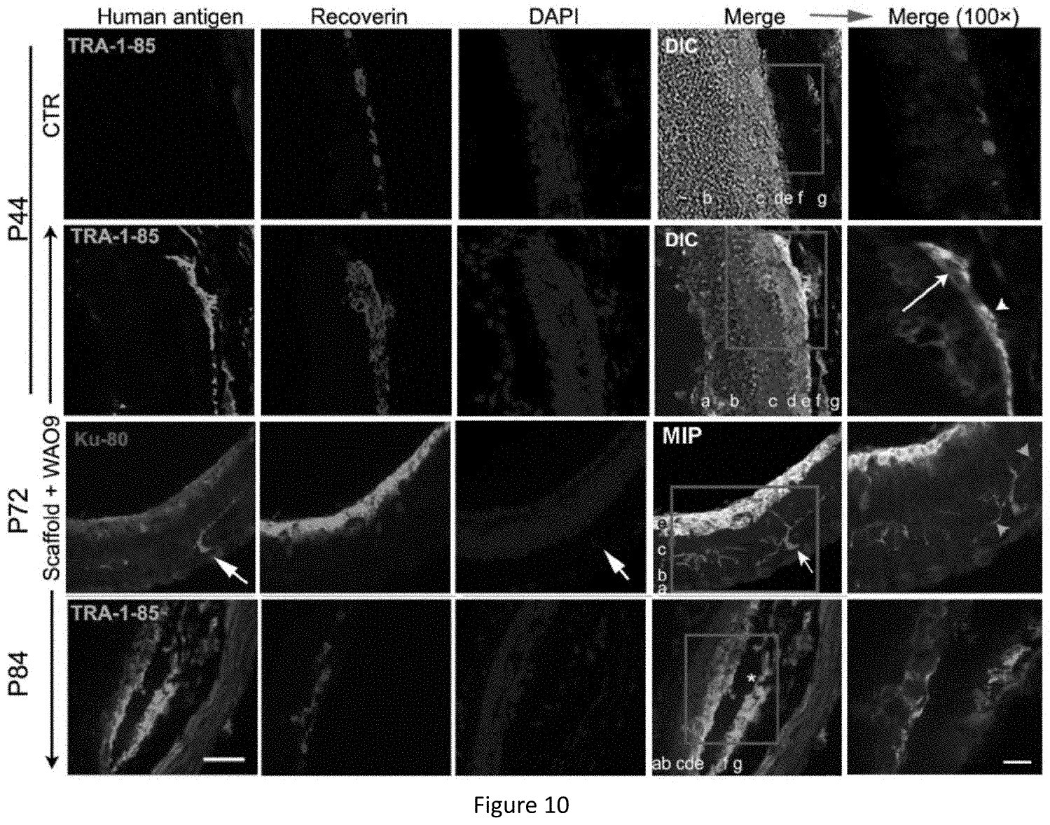

FIG. 24 demonstrates results from experiments 10 weeks post transplantation of GCH 521-RPC in the subretinal space of rd10 mice where immunohistological analysis was performed. Host bipolar cells were stained with PKC-a and human antigen with Ku80. Human antigen positive cells lined up the RPE layer (small arrows) and a faint label was associated with the ONL (large arrowheads), but nuclei were not labeled to indicate cytoplasmic transfer (see legend to FIG. 10). A few cells had cell bodies in the inner nuclear layer with processes that extended into the inner plexiform layer and co-labeled with PKC-α. Because the nuclei and cytoplasm were labeled, these are human cells that implanted into the host. Long arrow, background staining of the choroid, by the mouse monoclonal antibody for Ku-80. Bottom row scale Bar, 20 μm. Top row scale bar, 50 μm

FIG. 25 illustrates results from a functional recovery test that was performed for the 10 weeks of RD10 mice that received GCH-WA09-521 transplantation. The P1-wave response was recorded and temporal superior quadrant (site of implantation) showed better response to light stimulus in comparison to the remaining quadrants (color coded in the map, top right). Fundus image (top left) shows shadow of the scaffold. The top middle image shows the recordings without the fundus image. Analyzing each quadrant showed higher response in the red quadrant (Temporal and Nasal superior) side of the rd10 mice. The lower left shows averaged recordings that correspond to the colored map. Although recording for the green quadrant (tracing 1) appears normal, inspection of the individual tracings suggest noise and drift might account for the result. In contrast, the red recording has a similar shape to the un-averaged recordings. The lower right shows a three-dimensional graph of the P1 recordings with a peak centered on the graft.

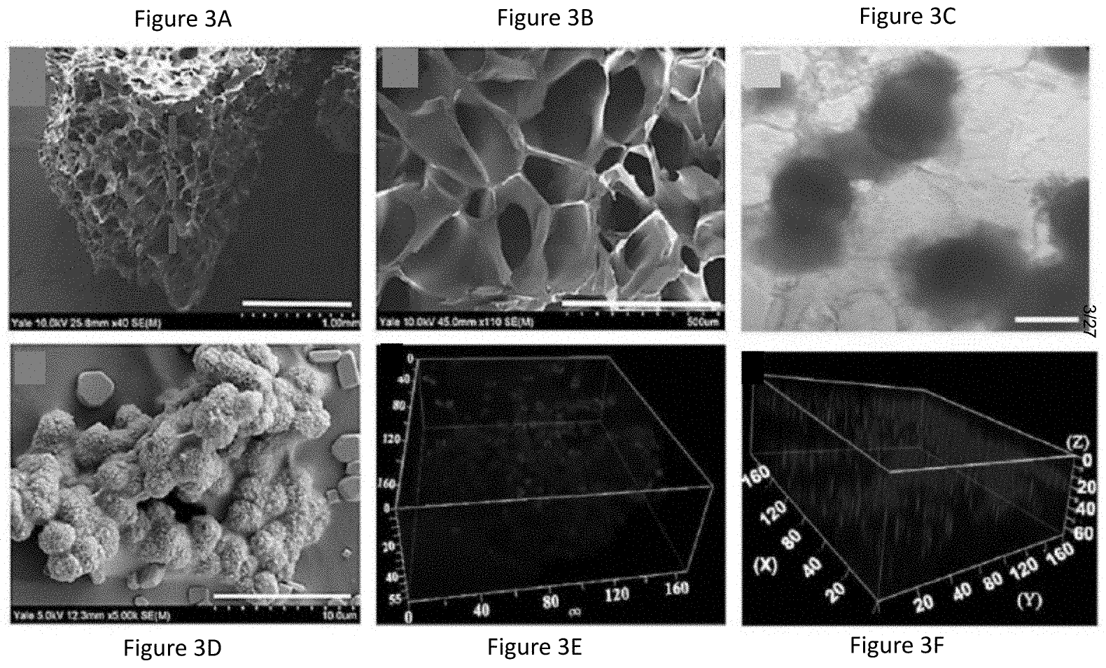

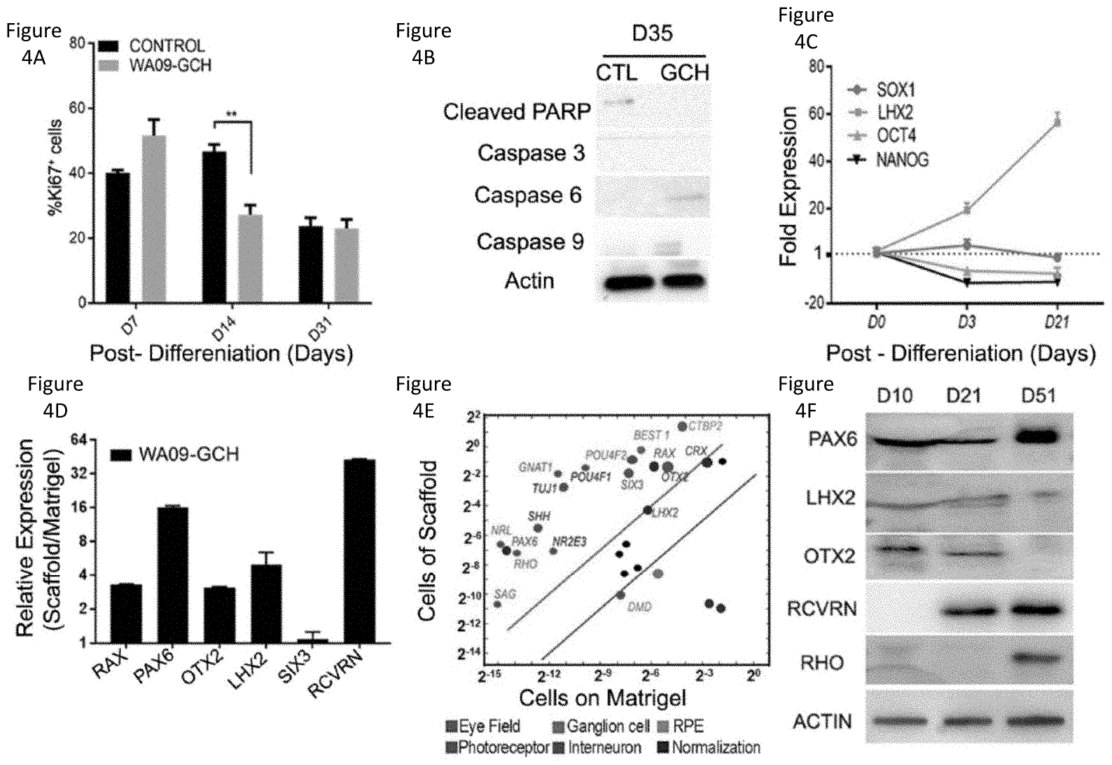

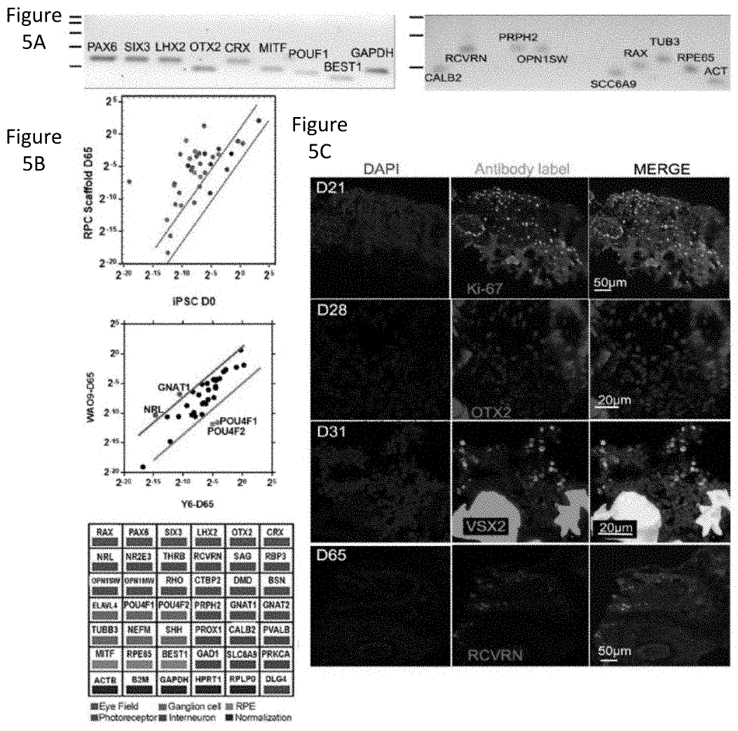

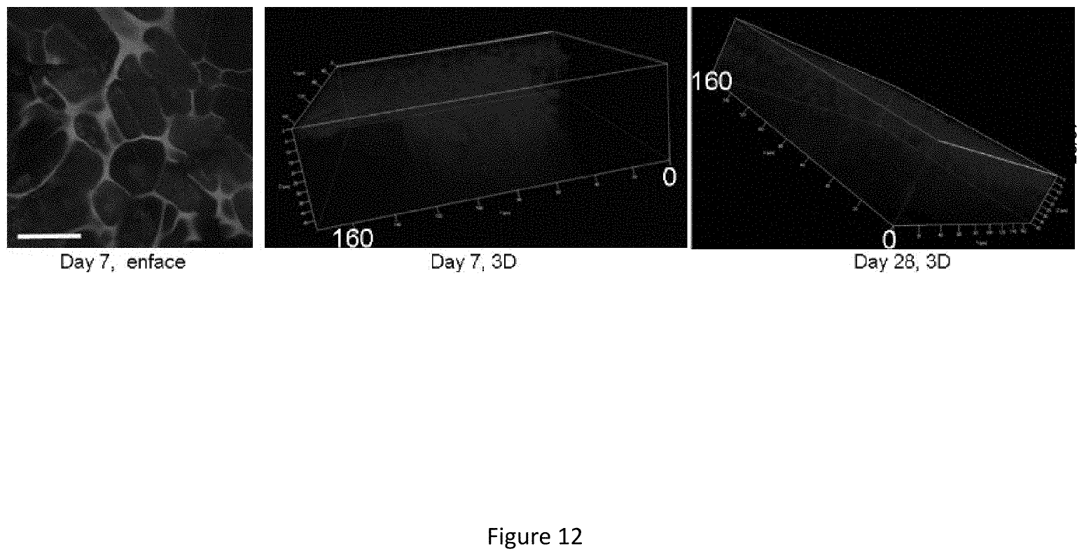

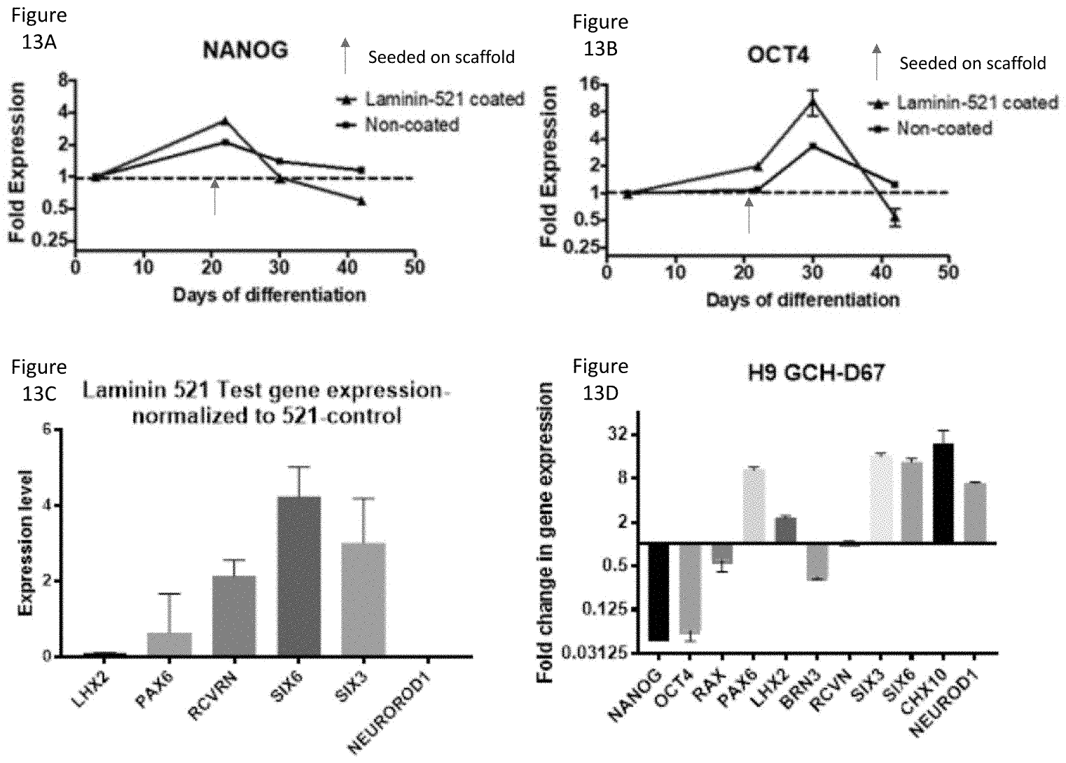

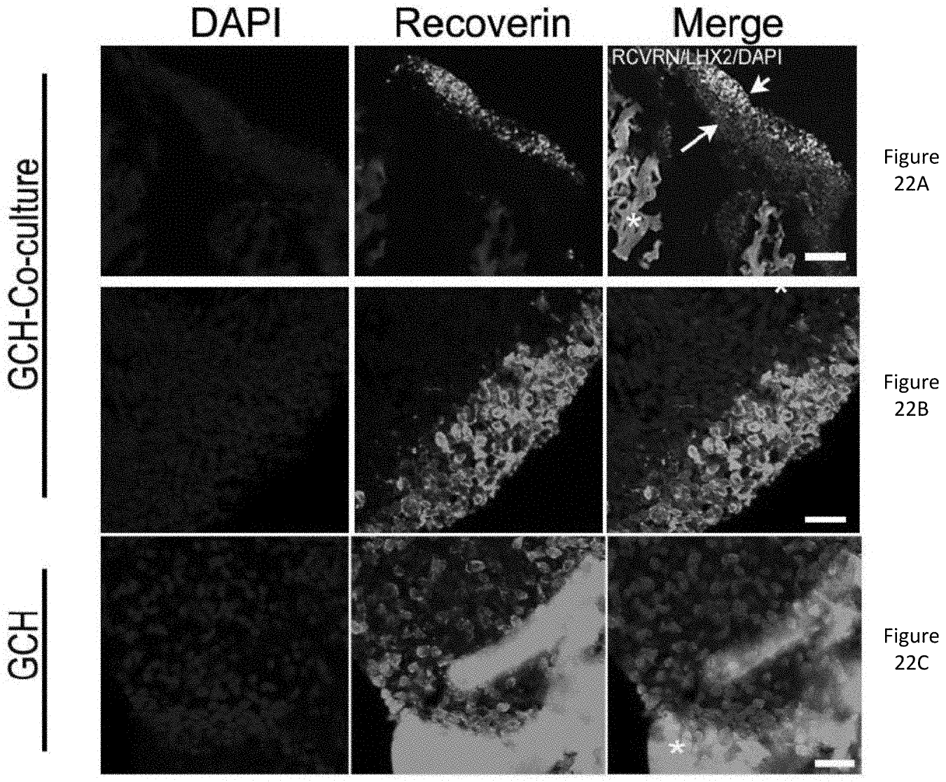

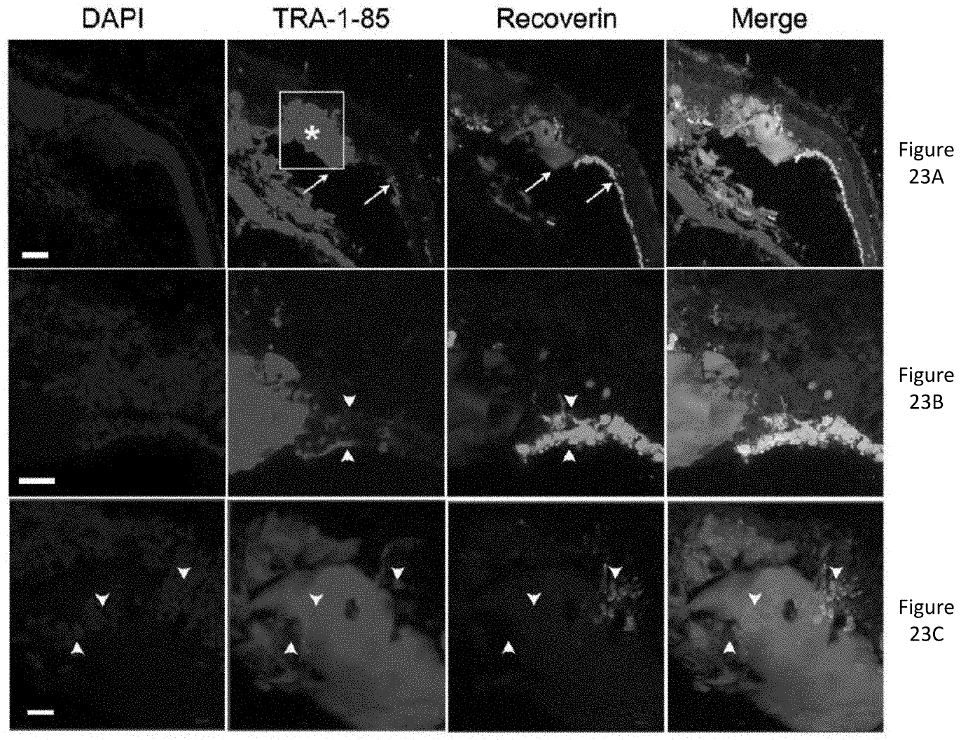

FIG. 26A through FIG. 26C depict the differentiation of RPC cultured on electrospun PCL scaffold was affected by co-culture with RPE. FIG. 26A depicts an exemplary scaffold, viewed en face by scanning electron microscopy showing loose, randomly oriented PCL fibers less than 5 μm in diameter. Scale bar, 500 μm. As depicted in FIG. 26B, cells migrated up to 40 microns into the 100 μm-thick scaffold, but not as effectively on the GCH scaffold, as compared with FIG. 3A-FIG. 3F and FIG. 12. (blue, non-proliferating cells; violet, proliferating cells). As depicted in FIG. 26C, after 3 weeks of co-culture, RPE and RPC were separate and analyzed for the expression of mRNA. Expression of photoreceptor markers increased as a result of co-culture, consistent with the data in FIG. 20C and FIG. 20D.

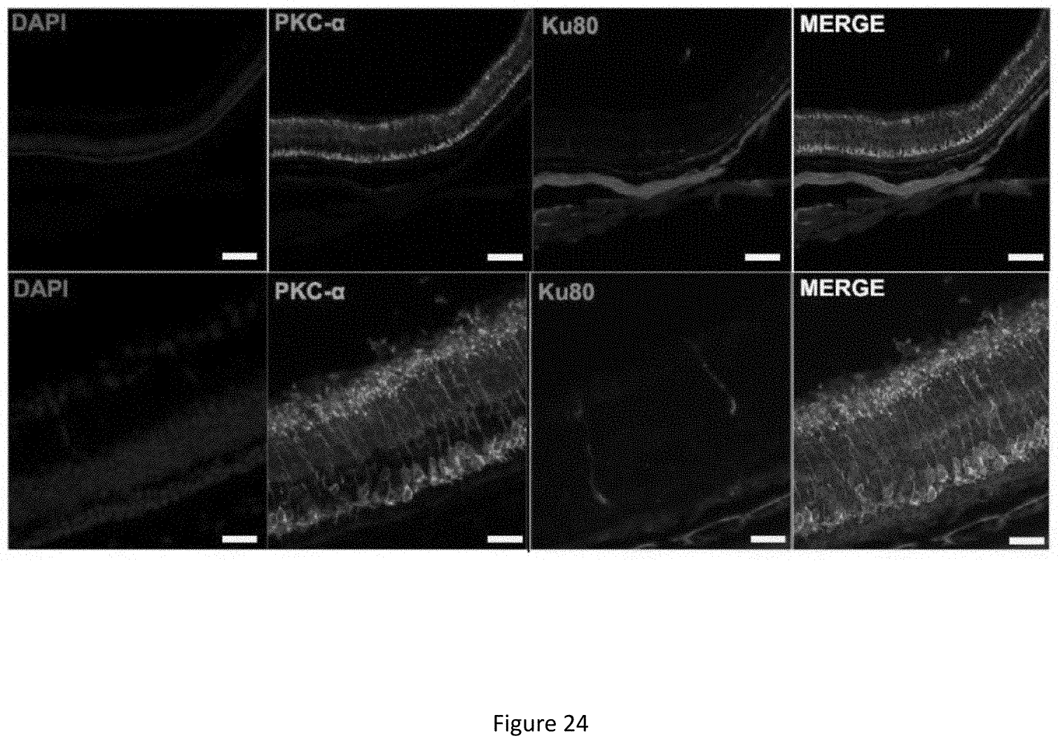

FIG. 27 depicts confocal images indicating BNDF increased the extension of ganglion cell neurites. RPC were cultured for 13 days on the scaffold before adding BNDF for 3 days to the vitreal medium chamber (FIG. 21A). The data demonstrate the feasibility of using the scaffold as a model to test putative pharmaceutical agents. Scale Bar, 20 μm.

### DETAILED DESCRIPTION

The present invention relates to a scaffold comprising extracellular matrix proteins for culturing specialized tissue for implantation into the eye and methods for doing the same. In some aspects, the invention also relates to methods for screening for therapeutic agents that modify the development of the tissues or cells of the retina, including retinal epithelial cells and nerve cells.

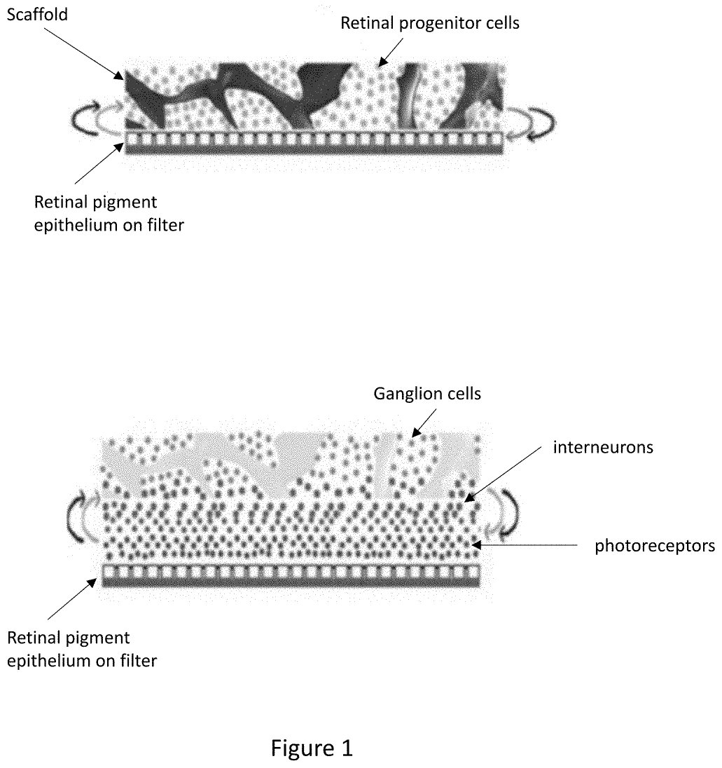

The theoretical underpinning of this invention is shown in FIG. 1. Stem cells or retinal precursors are seeded on a scaffold and layered on retinal pigment epithelium that sits on a filter (FIG. 1, top). The arrows indicate signals that are sent back and forth between the tissues to foster each other's differentiation and maturation. As the retinal precursors differentiate, the developing cells also send signals back and forth (FIG. 1, bottom). Both signaling processes occur simultaneously thereby resulting in a planar, multi-laminar neo-retina.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

As used here, “biocompatible” refers to any material, which, when implanted in a mammal, does not provoke an adverse response in the mammal. A biocompatible material, when introduced into an individual, is not toxic or injurious to that individual, nor does it induce immunological rejection of the material in the mammal.

As used herein, a “culture,” refers to the cultivation or growth of cells, for example, tissue cells, in or on a nutrient medium. As is well known to those of skill in the art of cell or tissue culture, a cell culture is generally begun by removing cells or tissue from a human or other animal, dissociating the cells by treating them with an enzyme, and spreading a suspension of the resulting cells out on a flat surface, such as the bottom of a Petri dish. There the cells generally form a thin layer of cells called a “monolayer” by producing glycoprotein-like material that causes the cells to adhere to the plastic or glass of the Petri dish. A layer of culture medium, containing nutrients suitable for cell growth, is then placed on top of the monolayer, and the culture is incubated to promote the growth of the cells.

The term “decellularized” or “decellularization” as used herein refers to a biostructure (e.g., an organ, or part of an organ), from which the cellular and tissue content has been removed leaving behind an intact acellular infra-structure. Organs such as the kidney are composed of various specialized tissues. The specialized tissue structures of an organ, or parenchyma, provide the specific function associated with the organ. The supporting fibrous network of the organ is the stroma. Most organs have a stromal framework composed of unspecialized connecting tissue which supports the specialized tissue. The process of decellularization removes the specialized tissue, leaving behind the complex three-dimensional network of connective tissue. The connective tissue infra-structure is primarily composed of collagen. The decellularized structure provides a biocompatible substrate onto which different cell populations can be infused. Decellularized biostructures may be rigid, or semi-rigid, having an ability to alter their shapes. Examples of decellularized organs useful in aspects of the present invention include, but are not limited to, the heart, kidney, liver, pancreas, spleen, bladder, ureter and urethra, cartilage, bone, brain, spine cord, peripheral nerve.

The term “derived from” is used herein to mean to originate from a specified source.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

An “effective amount” or “therapeutically effective amount” of a compound is that amount of compound which is sufficient to provide a beneficial effect to the subject to which the compound is administered.

As used herein “endogenous” refers to any material from or produced inside an organism, cell or system.

“Exogenous” refers to any material introduced from or produced outside an organism, cell, or system.

As used herein, “extracellular matrix composition” includes both soluble and non-soluble fractions or any portion thereof. The non-soluble fraction includes those secreted ECM proteins and biological components that are deposited on the support or scaffold. The soluble fraction includes refers to culture media in which cells have been cultured and into which the cells have secreted active agent(s) and includes those proteins and biological components not deposited on the scaffold. Both fractions may be collected, and optionally further processed, and used individually or in combination in a variety of applications as described herein.

As used herein, a “graft” refers to a cell, tissue or organ that is implanted into an individual, typically to replace, correct or otherwise overcome a defect. A graft may further comprise a scaffold. The tissue or organ may consist of cells that originate from the same individual; this graft is referred to herein by the following interchangeable terms: “autograft,” “autologous transplant,” “autologous implant” and “autologous graft”. A graft comprising cells from a genetically different individual of the same species is referred to herein by the following interchangeable terms: “allograft,” “allogeneic transplant,” “allogeneic implant,” and “allogeneic graft.” A graft from an individual to his identical twin is referred to herein as an “isograft,” a “syngeneic transplant,” a “syngeneic implant” or a “syngeneic graft.” A “xenograft,” “xenogeneic transplant,” or “xenogeneic implant” refers to a graft from one individual to another of a different species.

As used herein, a “growth factor” is a substance, such as a vitamin, nutrient, protein, or hormone, including, but are not limited to, growth hormone, erythropoietin, thrombopoietin, interleukin 3, interleukin 6, interleukin 7, macrophage colony stimulating factor, c-kit ligand/stem cell factor, osteoprotegerin ligand, insulin, insulin like growth factors, epidermal growth factor (EGF), fibroblast growth factor (FGF), nerve growth factor, ciliary neurotrophic factor, platelet derived growth factor (PDGF), transforming growth factor (TGF-beta), hepatocyte growth factor (HGF), and bone morphogenetic protein, basic fibroblast growth factor (bFGF), transforming growth factor-beta (TGF-β), pigment epithelial-derived factor (PEDF), vascular endothelial growth factor (VEGF), bone morphogenic proteins (BMPs), sonic hedgehog (Shh), Wnts, neurotrophic agents including BNDF, CTNF, and bone marrow mesenchymal stromal cells, other fibroblast growth factors, other epithelial growth factors, other nerve growth factors, and tissue inhibitors of metalloproteinases (TIMP).

The term “monolith” as used herein refers to a block or assembly of materials such as extracellular matrix materials or constituents that are assembled or arranged into a block or aggregate of material. A monolith can refer to a crosslinked solution of extracellular matrix constituents and can also refer to an electrospun block or aggregate of material such as extracellular matrix constituents.

“Proliferation” is used herein to refer to the reproduction or multiplication of similar forms, especially of cells. That is, proliferation encompasses production of a greater number of cells, and may be measured by, among other things, simply counting the numbers of cells, measuring incorporation of ³H-thymidine into the cell, and the like.

The terms “stem cell”, “embryonic stem cell” and “induced pluripotent stem cells” are used herein to refer to either a pluripotent, or lineage-uncommitted, progenitor cell, which is potentially capable of an unlimited number of mitotic divisions to either renew itself or to produce progeny cells which will differentiate into the desired cell type. The term “induced pluripotent stem cell” as used herein refers to a type of pluripotent stem cell that can be generated directly from adult cells. The term “embryonic stem cell” as used herein refers to pluripotent stem cells derived from the inner cell mass of a blastocyst.

As used herein, “scaffold” refers to a structure, comprising a biocompatible material that provides a surface suitable for adherence and proliferation of cells. A scaffold may further provide mechanical stability and support. A scaffold may be in a particular shape or form so as to influence or delimit a three-dimensional shape or form assumed by a population of proliferating cells. Such shapes or forms include, but are not limited to, films (e.g., a form with two-dimensions substantially greater than the third dimension), ribbons, cords, sheets, flat discs, cylinders, spheres, 3-dimensional amorphous shapes, etc.

As used herein, a “substantially purified” component is a component that is essentially free of other components. Thus, a substantially purified cell refers to a cell which has been purified from other cell types with which it is normally associated in its naturally-occurring state.

A “therapeutic” treatment is a treatment administered to a subject who exhibits signs or symptoms of a disease or disorder, for the purpose of diminishing or eliminating the frequency and/or severity of at least one of those signs or symptoms.

As used herein, “treating a disease or disorder” means reducing the frequency and/or severity with which at least one sign or symptom of the disease or disorder is experienced by a patient.

As used herein, “tissue engineering” refers to the process of generating a tissue ex vivo for use in tissue replacement or reconstruction. Tissue engineering is an example of “regenerative medicine,” which encompasses approaches to the repair or replacement of tissues and organs by incorporation of cells, gene or other biological building blocks, along with bioengineered materials and technologies.

As used herein, the terms “tissue grafting” and “tissue reconstructing” both refer to implanting a graft into an individual to treat or alleviate a tissue defect, such as a lung defect or a soft tissue defect.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention provides novel scaffolds comprising a plurality of extracellular matrix constituents that, in some embodiments, further comprise supplemented extracellular matrix constituents. The scaffolds of the present invention support the differentiation of planar retinoids for implantation into the subretinal space for use in retinal repair and replacement as a treatment for diseases and disorders of the eye. The scaffold comprising extracellular matrix constituents in combination with the supplemented extracellular matrix constituents supports the coculture of differentiated cells and undifferentiated cells, promotes the differentiation of undifferentiated cells, and rapidly degrades when implanted into the subretinal space.

### Scaffolds

In some embodiments, the scaffold comprises at least one extracellular matrix constituent. In some embodiments, the scaffold comprises at least two extracellular matrix constituents. In some embodiments, the scaffold comprises a plurality of extracellular matrix constituents. In some embodiments, the scaffold of the present invention comprises gelatin, collagen, chondroitin sulfate, and hyaluronic acid and/or combinations thereof. In some embodiments, the scaffold comprises modified collagen I. In some embodiments, the scaffold comprises gelatin, chondroitin sulfate, and hyaluronic acid. In some embodiments, the ratio of concentrations of gelatin, chondroitin sulfate, and hyaluronic acid is about 2:1:2.

In some embodiments, the scaffold of the present invention is assembled by forming a monolith of constituents. In some embodiments, the monolith is formed by crosslinking a solution of extracellular matrix constituents. In some embodiments, the monolith is formed by electrospinning extracellular matrix constituents. In some embodiments, the monolith is formed by other suitable means known in the art. In some embodiments, the monolith is lyophilized. In some embodiments, the monolith is frozen and lyophilized.

In some embodiments, the scaffold further comprises supplemented extracellular matrix constituents, for example laminin. In some embodiments, the supplemented extracellular matrix constituents comprise fibronectin, laminin, vitronectin, or, for example, arginylglycylaspartic acid (RGD) for surface modification, which promotes cell adhesion, proliferation, differentiation, and/or maturation. In some embodiments, the scaffold further comprises any polysaccharide, including glycosaminoglycans (GAGs), with suitable antigen binding properties, recognition sequences, viscosity, molecular mass and other desirable properties. Suitable glycosaminoglycans include any glycan (i.e., polysaccharide) comprising an unbranched polysaccharide chain with a repeating disaccharide unit, one of which is an amino sugar. These compounds as a class carry a high negative charge, are strongly hydrophilic, and are commonly called mucopolysaccharides. This group of polysaccharides includes heparin, heparan sulfate, chondroitin sulfate, dermatan sulfate, keratan sulfate, and hyaluronic acid. These GAGs are predominantly found on cell surfaces and in the extracellular matrix. Glycosaminoglycan is also intended to include any glycan (i.e., polysaccharide) containing predominantly monosaccharide derivatives in which an alcoholic hydroxyl group has been replaced by an amino group or other functional group such as sulfate or phosphate. An example of a glycosaminoglycan is poly-N-acetyl glycosaminoglycan, commonly referred to as chitosan. Exemplary polysaccharides that may be useful in the present invention include dextran, heparan, heparin, hyaluronic acid, alginate, agarose, carageenan, amylopectin, amylose, glycogen, starch, cellulose, chitin, chitosan and various sulfated polysaccharides such as heparan sulfate, chondroitin sulfate, dextran sulfate, dermatan sulfate, or keratan sulfate. In some embodiments, the scaffold comprises polycaprolactone (PCL), poly(DL-lactic-co-glycolic acid, poly(glycol) acid and poly (lactic) acid.

In some embodiments, the scaffold is embedded or conjugated with at least one factor that is released by diffusion or upon degradation. In various embodiments, the at least one factor includes, but are not limited to epidermal growth factor (EGF), platelet derived growth factor (PDGF), basic fibroblast growth factor (bFGF), transforming growth factor-beta (TGF-β), pigment epithelial-derived factor (PEDF), vascular endothelial growth factor (VEGF), bone morphogenic proteins (BMPs), sonic hedgehog (Shh), Wnts, neurotrophic agents including brain-derived neurotrophic factor (BDNF), ciliary neurotrophic factor (CTNF), and bone marrow mesenchymal stem or stromal cells, other fibroblast growth factors, other epithelial growth factors, other nerve growth factors, and tissue inhibitors of metalloproteinases (TIMP). Additional factors such as antibiotics, bacteriocides, fungicides, silver-containing agents, analgesics, and nitric oxide releasing compounds may also be incorporated into the scaffolds of the present invention.

In some embodiments, scaffolds are seeded with at least one cell. In various embodiments, the at least one cell includes, but is not limited to an embryoid body derived from a human embryonic stem cell (hESC), an embryoid body derived from a human inducible pluripotent stem cell (hiPSC), a retinoid progenitor cell (RPC), a retinal pigment epithelium cell (RPE), a fibroblast, a keratinocyte, an epithelial cell, an endothelial cell, a mesenchymal stromal cell, and a stem cell.

In some embodiments, the scaffold is modified with functional groups for incorporating at least one protein or compound such as a therapeutic agent using suitable means as understood in the art, for example covalently linking. In some embodiments, the at least one therapeutic agent that is linked to the scaffold includes, but is not limited to, analgesics, anesthetics, antifungals, antibiotics, anti-inflammatories, anthelmintics, antidotes, antiemetics, antihistamines, antihypertensives, antimalarials, antimicrobials, antipsychotics, antipyretics, antiseptics, antiarthritics, antituberculotics, antitussives, antivirals, cardioactive drugs, cathartics, chemotherapeutic agents, a colored or fluorescent imaging agent, corticoids (such as steroids), antidepressants, depressants, diagnostic aids, diuretics, enzymes, expectorants, hormones, hypnotics, minerals, nutritional supplements, parasympathomimetics, potassium supplements, radiation sensitizers, a radioisotope, sedatives, sulfonamides, stimulants, sympathomimetics, tranquilizers, urinary anti-infectives, vasoconstrictors, vasodilators, vitamins, xanthine derivatives, neuroprotective agents, and the like. The therapeutic agent may also be other small organic molecules, naturally isolated entities or their analogs, organometallic agents, chelated metals or metal salts, peptide-based drugs, or peptidic or non-peptidic receptor targeting or binding agents. It is contemplated that linkage of the therapeutic agent to the scaffold may be via a protease sensitive linker or other biodegradable linkage. Additional molecules which may be incorporated into the scaffold include, but are not limited to, vitamins and other nutritional supplements; glycoproteins; fibronectin; laminin; laminin-521; gelatin; collagen; chondroitin sulfate; hyaluronic acid; peptides and proteins; carbohydrates (both simple and/or complex); proteoglycans; antigens; oligonucleotides (sense and/or antisense DNA and/or RNA); antibodies (for example, to infectious agents, tumors, drugs or hormones); and gene therapy reagents.

In some embodiments, the present invention provides a scaffold constructed from extracellular matrix proteins comprising gelatin (including modified collagen-1), chondroitin sulfate, and hyaluronic acid or GCH. In some embodiments, the scaffold is porous. In some embodiments, the scaffolds are prepared by crosslinking extracellular matrix constituents. Examples of scaffolds formed from physical or chemical crosslinking of hydrophilic extracellular matrix constituents, include but are not limited to, hyaluronans, chitosans, alginates, collagen, dextran, pectin, carrageenan, polylysine, gelatin or agarose (see.: W. E. Hennink and C. F. van Nostrum, 2002, Adv. Drug Del. Rev. 54, 13-36 and A. S. Hoffman, 2002, Adv. Drug Del. Rev. 43, 3-12). These materials consist of high-molecular weight backbone chains made of linear or branched polysaccharides or polypeptides. Examples of scaffolds based on chemical or physical crosslinking synthetic polymers include but are not limited to (meth)acrylate-oligolactide-PEO-oligolactide-(meth)acrylate, poly(ethylene glycol) (PEO), poly(propylene glycol) (PPO), PEO-PPO-PEO copolymers (Pluronics), poly(phosphazene), poly(methacrylates), poly(N-vinylpyrrolidone), PL(G)A-PEO-PL(G)A copolymers, poly(ethylene imine), etc. (see A. S Hoffman, 2002 Adv. Drug Del. Rev, 43, 3-12). In some embodiments, the hydrogel comprises poly(ethylene glycol) diacrylate (PEGDA).

In some embodiments, the scaffolds comprise a curing agent which initiates polymerization. For example, the scaffolds may comprise the photoinitiator 4-(2-hydroxyethoxy)phenyl-(2-hydroxy-2-propyl)ketone. In one embodiment, polymerization is induced by 4-(2-hydroxyethoxy)phenyl-(2-hydroxy-2-propyl)ketone upon application of UV light. Other examples of UV sensitive curing agents include 2-hydroxy-2-methyl-1-phenylpropan-2-one, 4-(2-hydroxyethoxy)phenyl (2-hydroxy-2-phenyl-2-hydroxy-2-propyl)ketone, 2,2-dimethoxy-2-phenyl-acetophenone 1-[4-(2-Hydroxyethoxy)-phenyl]-2-hydroxy-2-methyl-1-propane-1-one, 1-hydroxycyclohexylphenyl ketone, trimethyl benzoyl diphenyl phosphine oxide and mixtures thereof. The polymerization may be initiated by any suitable means in the art, such as by ultraviolet light or visible light. In certain embodiments, one or more multifunctional cross-linking agents may be utilized as reactive moieties that covalently link extracellular matrix constituents or synthetic polymers. Such bifunctional cross-linking agents may include glutaraldehyde, epoxides (e.g., bis-oxiranes), oxidized dextran, p-azidobenzoyl hydrazide, N-[α.-maleimidoacetoxy]succinimide ester, p-azidophenyl glyoxal monohydrate, bis-[β-(4-azidosalicylamido)ethyl]disulfide, bis[sulfosuccinimidyl]suberate, dithiobis[succinimidyl proprionate, disuccinimidyl suberate, 1-ethyl-3-[3-dimethylaminopropyl]carbodiimide hydrochloride (EDC), N-hydroxysuccinimide (NETS) and other bifunctional cross-linking reagents known to those skilled in the art. It should be appreciated by those in skilled in the art that the mechanical properties of the scaffold are greatly influenced by the cross-linking time and the amount of cross-linking agents.

The stabilized cross-linked scaffold of the present invention may be further stabilized and enhanced through the addition of one or more enhancing agents. By “enhancing agent” or “stabilizing agent” is intended any compound added to the scaffold, in addition to the high molecular weight components, that enhances the scaffold by providing further stability or functional advantages. Suitable enhancing agents, which are admixed with the high molecular weight components and dispersed within the scaffold, include many of the additives described earlier in connection with the thermoreversible scaffold discussed above. The enhancing agent may include any compound, especially polar compounds, that, when incorporated into the cross-linked scaffold, enhance the scaffold by providing further stability or functional advantages.

Enhancing agents for use with the stabilized cross-linked scaffold include polar amino acids, amino acid analogues, amino acid derivatives, intact collagen, and divalent cation chelators, such as ethylenediaminetetraacetic acid (EDTA) or salts thereof. Polar amino acids are intended to include tyrosine, cysteine, serine, threonine, asparagine, glutamine, aspartic acid, glutamic acid, arginine, lysine, and histidine. Exemplary polar amino acids include L-cysteine, L-glutamic acid, L-lysine, and L-arginine. Suitable concentrations of each particular enhancing agent are the same as noted above in connection with the thermoreversible hydrogel scaffold. Polar amino acids, EDTA, and mixtures thereof, are exemplary enhancing agents. The enhancing agents may be added to the scaffold composition before or during the crosslinking of the high molecular weight components.

In some embodiments, the scaffolds and/or scaffold monoliths of the present invention are frozen and lyophilized. In some embodiments, the lyophilized scaffold monoliths are sectioned. In some embodiments, the scaffold monoliths are sectioned in to planar sheets. In some embodiments, the monolith is sectioned into planar scaffold sheets comprising a thickness of about 60 μm. In some embodiments, the monolith is sectioned into planar scaffold sheets comprising about a thickness of 40 μm to about 80 μm. In some embodiments, the scaffold monolith is sectioned into planar scaffold sheets comprising a thickness of about 20 μm to about 100 μm.

In some embodiments, the scaffolds of the present invention are prepared by electrospinning extracellular matrix constituents. In some embodiments, the scaffolds of the present invention are prepared by incorporating extracellular matrix constituents into nanofibrous biocompatible electrospun matrices. Electrospinning is an atomization process of a conducting fluid which exploits the interactions between an electrostatic field and the conducting fluid. When an external electrostatic field is applied to a conducting fluid (e.g., a semi-dilute polymer solution or a polymer melt), a suspended conical droplet is formed, whereby the surface tension of the droplet is in equilibrium with the electric field. Electrostatic atomization occurs when the electrostatic field is strong enough to overcome the surface tension of the liquid. The liquid droplet then becomes unstable and a tiny jet is ejected from the surface of the droplet. As it reaches a grounded target, the material may be collected as an interconnected web containing relatively fine, i.e., small diameter, fibers. The resulting films (or membranes) from these small diameter fibers have very large surface area to volume ratios and small pore sizes. A detailed description of electrospinning apparatus is provided in Zong, et al., 2002 Polymer 43: 4403-4412; Rosen et al., 1990 Ann Plast Surg 25: 375-87; Kim, K., Biomaterials 2003, 24: 4977-85; Zong, X., 2005 Biomaterials 26: 5330-8. After electrospinning, extrusion and molding may be utilized to further fashion the polymers. To modulate fiber organization into aligned fibrous polymer scaffolds, the use of patterned electrodes, wire drum collectors, or post-processing methods such as uniaxial stretching has been successful (Zong, X., 2005 Biomaterials 26: 5330-8; Katta, P., 2004 Nano Lett 4: 2215-2218; Li, D., 2005 Nano Lett 5: 913-6).

In some embodiments, the scaffold comprising extracellular matrix constituents is produced in one of several ways. In one embodiment, the method involves adding a solution comprising extracellular matrix constituents to an appropriate solvent. In some embodiments, this process is accomplished in a syringe assembly or it is subsequently loaded into a syringe assembly. In some embodiments, the method involves purchasing commercially available polymer solutions or commercially available polymers and dissolving them to create polymer solutions. For example, poly(ethylene oxide) (PEO) is available from Sigma (Sigma, St. Louis, Mo.), poly-L-lactide (PLLA) is available from DuPont (Wilmington, Del.), poly(lactide-co-glycolide) is available from Ethicon (Somerville, N.J.). Additional polymer scaffold components of the invention, such as cells and biomolecules, are also commercially available from suppliers.

In some embodiments, the solution comprising extracellular matrix constituents used to form the scaffold is first dissolved in a solvent. In some embodiments, the solvent is any solvent which is capable of dissolving the extracellular matrix constituents. Typical solvents include N,N-Dimethyl formamide (DMF), tetrahydrofuran (THF), methylene chloride, dioxane, ethanol, hexafluoroisopropanol (HFIP), chloroform, 1,1,1,3,3,3-hexafluoro-2-propanol (HFP), glacial acetic acid, water, and combinations thereof.

In some embodiments, the solution comprising extracellular matrix constituents contain a salt which creates an excess charge effect to facilitate the electrospinning process. Examples of suitable salts include NaCl, KH₂PO₄, K₂HPO₄, KIO₃, KCl, MgSO₄, MgCl₂, NaHCO₃, CaCl₂ or mixtures of these salts.

In some embodiments, the solution forming the conducting fluid has a protein concentration in the range of about 1 to about 80 wt %, or about 8 to about 60 wt %.

In some embodiments, the electric field created in the electrospinning process is in the range of about 5 to about 100 kilovolts (kV), or about 10 to about 50 kV. In some embodiments, the feed rate of the conducting fluid to the spinneret (or electrode) is in the range of about 0.1 to about 1000 microliters/min, or about 1 to about 250 microliters/min. The single or multiple spinnerets sit on a platform which is capable of being adjusted, varying the distance between the platform and the grounded collector substrate.

The distance may be any distance which allows the solvent to essentially completely evaporate prior to the contact of the polymer with the grounded collector substrate. In an exemplary embodiment, this distance may vary from 1 cm to 25 cm. Increasing the distance between the grounded collector substrate and the platform generally produces thinner fibers.

In electrospinning cases where a rotating mandrel is required, the mandrel is mechanically attached to a motor, often through a drill chuck. In an exemplary embodiment, the motor rotates the mandrel at a speed of between about 1 revolution per minute (rpm) to about 500 rpm. In an exemplary embodiment, the motor rotation speed of between about 200 rpm to about 500 rpm. In another exemplary embodiment, the motor rotation speed of between about 1 rpm to about 100 rpm.

The invention also includes combinations of natural materials, combinations of synthetic materials, and combinations of both natural and synthetic materials. For example, the extracellular matrix constituents of the invention may be combined with natural materials, synthetic materials, or both natural and synthetic materials to produce the scaffolds of the invention. Examples of combinations include, but are not limited to: blends of different types of collagen (e.g. Type I with Type II, Type I with Type III, Type II with Type III, etc.); blends of one or more types of collagen with fibrinogen, thrombin, gelatin, chondroitin sulfate, hyaluronic acid, elastin, PGA, PLA, alginate, and polydioxanone; blends of one or more types of collagen (e.g. collagen and gelatin) with chondroitin sulfate, hyaluronic acid; blends of blends of one or more types of collagen (e.g. collagen and gelatin) with chondroitin sulfate, hyaluronic acid and laminin (e.g. laminin 521); and blends of fibrinogen with one or more types of collagen, thrombin, elastin, PGA, PLA, and polydioxanone.

In some embodiments, the electroprocessed material of the present invention results from the electroprocessing of natural materials, synthetic materials, or combinations thereof. Examples include but are not limited to amino acids, peptides, denatured peptides such as gelatin from denatured collagen, polypeptides, proteins, carbohydrates, lipids, nucleic acids, glycoproteins, lipoproteins, glycolipids, glycosaminoglycans, and proteoglycans.

In various embodiments, materials to be electroprocessed are naturally occurring extracellular matrix materials and blends of naturally occurring extracellular matrix materials, including but not limited to collagen, gelatin, fibrin, fibrinogen, thrombin, elastin, laminin, fibronectin, hyaluronic acid, chondroitin 4-sulfate, chondroitin 6-sulfate, dermatan sulfate, heparin sulfate, heparin, and keratan sulfate, and proteoglycans. In other embodiments, materials for electroprocessing include collagen, fibrin, fibrinogen, thrombin, fibronectin, and combinations thereof. Some collagens that are used include but are not limited to collagen types I, II, III, IV, V, VI, VII, VIII, IX, X, XI, XII, XIII, XIV, XV, XVI, XVII, XVIII, and XIX. In some embodiments, collagens include types I, II, and III. These proteins may be in any form, including but not limited to native and denatured forms. In some embodiments, materials for electroprocessing are carbohydrates such as polysaccharides (e.g., cellulose and its derivatives), chitin, chitosan, alginic acids, and alginates such as calcium alginate and sodium alginate. In some embodiments, these materials are isolated from plant products, humans or other organisms or cells or synthetically manufactured. In some embodiments, the natural material for electroprocessing includes at least one of collagen, fibrinogen, thrombin, fibrin, fibronectin, gelatin, chondroitin sulfate, hyaluronic acid, and laminin. In some embodiments, the natural material for electroprocessing may also include a crude extract of tissue, extracellular matrix material, an extract of non-natural tissue, or extracellular matrix materials (i.e., extracts of cancerous tissue), alone or in combination. Extracts of biological materials, including but are not limited to cells, tissues, organs, and tumors may also be electroprocessed.

The invention includes all natural or natural-synthetic hybrid compositions that result from the electroprocessing of any material. Materials that change in composition or structure before, during, or after electroprocessing are within the scope of the invention.

It is to be understood that these electroprocessed materials may be combined with other materials and/or substances in forming the compositions of the present invention. Electroprocessed materials in some embodiments are prepared at very basic or acidic pHs (for example, by electroprocessing from a solution having a specific pH) to accomplish the same effect. As another example, an electroprocessed scaffold, containing cells, may be combined with an electroprocessed biologically compatible polymer to stimulate growth and division of the cells in the electroprocessed scaffold.

In various embodiments, synthetic materials electroprocessed for use in the scaffold include any materials prepared through any method of artificial synthesis, processing, isolation, or manufacture. In some embodiments, the synthetic materials are biologically compatible for administration in vivo or in vitro. In various embodiments, synthetic materials comprise polymers which may include but are not limited to the following: poly(urethanes), poly(siloxanes) or silicones, poly(ethylene), poly(vinyl pyrrolidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactic acid (PLA), polyglycolic acids (PGA), poly(lactide-co-glycolides) (PLGA), nylons, polyamides, polyanhydrides, poly(ethylene-co-vinyl alcohol) (EVOH), polycaprolactone, poly(vinyl acetate) (PVA), polyvinylhydroxide, poly(ethylene oxide) (PEO) and polyorthoesters or any other similar synthetic polymers that may be developed that are biologically compatible. In some embodiments, synthetic materials include PLA, PGA, copolymers of PLA and PGA, polycaprolactone, poly(ethylene-co-vinyl acetate), EVOH, PVA, and PEO. In some embodiments, the polymers have cationic, including, but are not limited to, poly(allyl amine), poly(ethylene imine), poly(lysine), and poly(arginine). The polymers may have any molecular structure including, but not limited to, linear, branched, graft, block, star, comb and dendrimer structures. Matrices may be formed of electrospun fibers, electroaerosol, electrosprayed, or electrosputtered droplets, electroprocessed powders or particles, or a combination of the foregoing.

By selecting different natural and synthetic materials, or combinations thereof, many characteristics of the scaffold are manipulated. The properties of the scaffold comprised electroprocessed material and a substance may be adjusted. In some embodiments, selection of materials for electroprocessing affects the permanency of an implanted scaffold. For example, many scaffolds made by electroprocessing fibrinogen or fibrin may degrade more rapidly while many scaffolds made of collagen are more durable and many other scaffolds made by electroprocessing materials are more durable still. Thus, for example, incorporation of durable synthetic polymers (e.g., PLA, PGA) increase the durability and structural strength of scaffolds electroprocessed from solutions of fibrinogen in some embodiments. Use of scaffolds made by electroprocessing natural materials such as proteins derived from corn, wheat, potato, sorghums, tapioca, rice, arrow root, sago, soybean, pea, sunflower, peanut, gelatin, and the like also minimize rejection or immunological response to an implanted scaffold. Accordingly, selection of materials for electroprocessing and use in substance delivery is influenced by the desired use.

In some embodiments in which the scaffold contains substances that are to be released from the scaffold, incorporating electroprocessed synthetic components, such as biocompatible substances, modulates the release of substances from an electroprocessed composition. For example, layered or laminate structures may be used to control the substance release profile. Unlayered structures may also be used, in which case the release is controlled by the relative stability of each component of the construct. For example, layered structures composed of alternating electroprocessed materials are prepared by sequentially electroprocessing different materials onto a target. The outer layers are, for example, tailored to dissolve faster or slower than the inner layers. Multiple agents may be delivered by this method, optionally at different release rates. Layers may be tailored to provide a complex, multi-kinetic release profile of a single agent over time. Using combinations of the foregoing provides for release of multiple substances released, each with its own profile. Complex profiles are possible.

In some embodiments, natural components such as biocompatible substances are used to modulate the release of electroprocessed materials or of substances from an electroprocessed composition. For example, a drug or series of drugs or other materials or substances to be released in a controlled fashion may be electroprocessed into a series of layers. In one embodiment, one layer is composed of electroprocessed fibrinogen plus a drug, the next layer PLA plus a drug, a third layer is composed of polycaprolactone plus a drug. The layered construct may be implanted, and as the successive layers dissolve or break down, the drug (or drugs) is released in turn as each successive layer erodes. In some embodiments, unlayered structures are used, and release is controlled by the relative stability of each component of the construct.

### Methods of Assembling Scaffolds

In some embodiments, the scaffolds of the present invention, comprising crosslinked scaffolds and electrospun scaffolds, are prepared using means known in the art as described herein to form a monolith. In some embodiments, the scaffold monoliths of the present invention may or may not be frozen using means as known by those skilled in the art. In some embodiments, the scaffold monoliths of the present invention may or may not be lyophilized using means as known by those skilled in the art. In some embodiments, the lyophilized scaffolds are sectioned. In some embodiments, the scaffolds are sectioned into planar sheets. In some embodiments, the scaffolds of the present invention are sectioned without being lyophilized. In some embodiments, the scaffold monolith is sectioned into planar sheets comprising a thickness of about 60 μm. In some embodiments, the scaffold monolith is sectioned into planar sheets comprising about a thickness of about 40 μm to about 80 μm. In some embodiments, the scaffold is sectioned into planar sheets comprising a thickness of about 20 μm to about 100 μm.

In some embodiments, the scaffolds of the present invention, which in some embodiments, are sectioned planar sheets of scaffold monoliths as described herein are layered on top of a monolayer of cultured cells. In some embodiments, the cultured cells are mature cells, for example mature epithelial cells including retinal pigment epithelial cells. In some embodiments, the cells are progenitor cells. In some embodiments, the cells are stem cells. In some embodiments, the progenitor cells are retinal progenitor cells. In some embodiments, the cells are embryoid bodies that in some embodiments are derived from human embryonic stem cells (hESC), human inducible pluripotent stem cells (hiPSC), or retinoid progenitor cells (RPC).

In some embodiments, the planar sheet of the sectioned scaffold monolith is seeded with at least one cell. In some embodiments, the at least one cell is a mature cell. In some embodiments, the at least one cell is a progenitor cell. In some embodiments, the at least one cell is a stem cell. In some embodiments, the progenitor cell is a retinal progenitor cell. In some embodiments, the cells are embryoid bodies derived from an hESC, hiPSC, or RPC. In some embodiments, the scaffold promotes the differentiation of at least one progenitor cell into a mature differentiated cell such as a retinal epithelial cell. In some embodiments, the scaffold promotes the differentiation of a progenitor cell into an organoid such as a retinal organoid.

In some embodiments, the scaffold is seeded with one or more populations of cells to form an artificial organ construct such as an artificial retinal tissue. The artificial organ construct may be autologous (where the cell populations are derived from the subject's own tissue), or allogenic (where the cell populations are derived from another subject within the same species as the patient). The artificial organ construct may also be xenogenic, where the one or more populations of cells populations are derived form a mammalian species that is different than the subject. In various embodiments, the cells are derived from a mammal such as a human, a monkey, a dog, a cat, a mouse, a rat, a cow, a horse, a pig, a goat and a sheep.

In various embodiments, the cell type includes, but is not limited to, a retinal pigment epithelial cell, a retinal progenitor cell, an inducible pluripotent stem cell, an embryonic stem cell, a bone marrow derived stem cell, a bipolar cell, a Muller cell, an amacrine cell, a retinal ganglion cell, a nerve cell, a mesenchymal cell, such as a smooth or skeletal muscle cell, a myocytes (muscle stem cells), a fibroblast, a chondrocyte, an adipocyte, a fibromyoblast, an ectodermal cell, including ductile and skin cells, a hepatocyte, an Islet cell, a cell present in the intestine and other parenchymal cells, and an osteoblasts or other cell forming bone or cartilage.

Isolated cells may be cultured in vitro to increase the number of cells available for coating the scaffold. The use of allogenic cells, or autologous cells, is useful for preventing tissue rejection. However, if an immunological response does occur in the subject after implantation of the artificial organ, the subject may be treated with immunosuppressive agents such as, cyclosporin or FK506, to reduce the likelihood of rejection. In certain embodiments, chimeric cells, or cells from a transgenic animal, are coated onto the biocompatible scaffold.

In some embodiments, cells are transfected with genetic material prior to seeding onto the scaffold. Useful genetic material includes, for example, genetic sequences which are capable of reducing or eliminating an immune response in the host. For example, the expression of cell surface antigens such as class I and class II histocompatibility antigens may be suppressed. In some embodiments, this allows the transplanted cells to have reduced chance of rejection by the host. In addition, transfection could also be used for gene delivery.

In some embodiments, cells are normal or genetically engineered to provide additional or normal function. In some embodiments, methods for genetically engineering cells with retroviral vectors, polyethylene glycol, or other methods known to those skilled in the art are used. These include using expression vectors which transport and express nucleic acid molecules in the cells. (See Goeddel; Gene Expression Technology: Methods in Enzymology 185, Academic Press, San Diego, Calif. (1990).

Vector DNA is introduced into cells via conventional transformation or transfection techniques. Suitable methods for transforming or transfecting host cells can be found in Sambrook et al. (Molecular Cloning: A Laboratory Manual, 3nd Edition, Cold Spring Harbor Laboratory press (2001)), and other laboratory textbooks.

In some embodiments, seeding of cells onto the matrix or scaffold is performed according to standard methods. For example, the seeding of cells onto polymeric substrates for use in tissue repair has been reported (see, e.g., Atala, A. et al., J. Urol. 148(2 Pt 2): 658-62 (1992); Atala, A., et al. J. Urol. 150 (2 Pt 2): 608-12 (1993)). Cells grown in culture may be trypsinized to separate the cells, and the separated cells may be seeded on the scaffold. Alternatively, cells obtained from cell culture are lifted from a culture plate as a cell layer, and the cell layer is directly seeded onto the scaffold without prior separation of the cells.

In some embodiments, in the range of 1 million to 50 million cells are suspended in medium and applied to each square centimeter of a surface of a scaffold. In some embodiments, between 1 million and 50 million cells, and in some embodiments, between 1 million and 10 million cells are suspended in media and applied to each square centimeter of a surface of a scaffold. The matrix or scaffold is incubated under standard culturing conditions, such as, for example, 37° C., 5% CO₂, for a period of time until the cells attach. However, it will be appreciated that the density of cells seeded onto the scaffold may be varied. For example, greater cell densities promote greater tissue regeneration by the seeded cells, while lesser densities may permit relatively greater regeneration of tissue by cells infiltrating the graft from the host. In some embodiments, other seeding techniques are used depending on the matrix or scaffold and the cells. For example, in some embodiments, the cells are applied to the matrix or scaffold by vacuum filtration. Selection of cell types, and seeding of cells onto a scaffold, will be routine to one of ordinary skill in the art in light of the teachings herein.

In some embodiments, the scaffold is seeded with one population of cells to form an artificial organ construct. In some embodiments, the scaffold is seeded with one population of cells and placed in contact with another population of cells such that in some embodiments, the scaffold is used to support the co-culture of two or more populations of cells. In another embodiment, the scaffold is seeded on two sides with two different populations of cells. In some embodiments, this is performed by first seeding one side of the scaffold and then seeding the other side. For example, the scaffold is then placed with one side on top and seeded. In some embodiments, the scaffold is repositioned so that a second side is on top. In some embodiments, the second side is then seeded with a second population of cells. Alternatively, both sides of the scaffold may be seeded at the same time. For example, in some embodiments, two cell chambers are positioned on both sides (i.e., a sandwich) of the scaffold. In some embodiments, the two chambers are filled with different cell populations to seed both sides of the scaffold simultaneously. In some embodiments, the sandwiched scaffold is rotated, or flipped frequently to allow equal attachment opportunity for both cell populations. In some embodiments, simultaneous seeding is prepared when the pores of the scaffold are sufficiently large for cell passage from one side to the other side. In some embodiments, seeding the scaffold on both sides simultaneously reduces the likelihood that the cells would migrate to the opposite side. In some embodiments, the cells are any suitable cell type that may communicated with one or more other populations of cells through direct physical contact such as via cell-cell contact, junctions, and the like. In some embodiments, the one or more populations of cells are positioned in proximity with each other such that they may communicate by secreting factors such as paracrine, endocrine, or autocrine factors that may direct growth, differentiation, migration and the like.

In another embodiment, two separate scaffolds may be seeded with different cell populations. In some embodiments, after seeding, the two matrices are attached together to form a single scaffold with two different cell populations on the two sides. In some embodiments, attachment of the scaffolds to each other is performed using standard procedures such as fibrin glue, liquid co-polymers, sutures and the like.

In order to facilitate cell growth on the scaffold of the present invention, the scaffold may be coated with one or more cell adhesion-enhancing agents. These agents include but are not limited collagen, laminin, for example laminin-521, and fibronectin. The scaffold may also contain cells cultured on the scaffold to form a target tissue substitute. The target tissue that may be formed using the scaffold of the present invention may be retinal tissue. In some embodiments, the present invention provides methods for generating a scaffold for culturing retinal tissue comprising an amount of gelatin, an amount of chondroitin sulfate, an amount of hyaluronic acid, wherein the amount of gelatin, chondroitin sulfate, and hyaluronic acid are prepared into a three-dimensional monolith, wherein the monolith is sectioned into planar sheets, and an amount of laminin-521. In some embodiments, the scaffold is then seeded with retinal progenitor cells, and placed in direct contact with a monolayer of retinal pigment epithelial cells; thereby creating a coculture assembly. In some embodiments, the coculture assembly is incubated under appropriate conditions including using appropriate media compositions and appropriate environmental conditions (i.e., 37° C., 5% CO₂), thereby generating an organoid. In some embodiments, the present invention further comprises methods for implanting the generated organoid into the subretinal space of a subject.

While the scaffolds of the present invention are stable and have a long shelf life, in some embodiments it is advantageous to include one or more preservative. The preservative may comprise from about 0.005% to 2.0% by total weight of the scaffold. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof.

In some embodiments, the scaffold includes an anti-oxidant and a chelating agent that inhibits the degradation of one or more components of extracellular matrix constituents. In some embodiments, the antioxidants for some compounds are butylated hydroxytoluene (BHT), butylated hydroxyanisole (BHA), alpha-tocopherol and ascorbic acid. In some embodiments, antioxidants are included in the range of about 0.01% to 0.3%. In some embodiments, BHT is included in the range of 0.03% to 0.1% by weight by total weight of the composition. In some embodiments, the chelating agent is present in an amount of from 0.01% to 0.5% by weight by total weight of the scaffold. In some embodiments, chelating agents include edetate salts (e.g., disodium edetate) and citric acid in the weight range of about 0.01% to 0.20% and in some embodiments, in the range of 0.02% to 0.10% by weight by total weight of the scaffold. The chelating agent is useful for chelating metal ions in the scaffold that may be detrimental to the shelf life of the formulation. While in some embodiments, BHT and disodium edetate are the antioxidant and chelating agent respectively for some compounds, in some embodiments, other suitable and equivalent antioxidants and chelating agents are substituted therefore as would be known to those skilled in the art.

### Screening Tool

In some embodiments, the present invention provides a platform for screening for therapeutic agents as described herein that may regulate the growth, regeneration, function and/or differentiation of retinal cells and retinal tissue. Many retinal degenerations begin with the retinal pigment epithelium or the photoreceptors, but the common result is the outer nuclear layer dies and signal inputs to interneurons and ganglion cells are lost. Lacking signal inputs, synapses between these cells disappear, neurites are withdrawn, and cell death slowly increases. The present invention provides a platform for screening for neuroprotection therapies, for example neuroprotection therapies that may promote maintenance of interneuron and ganglion cells prior to, and after, surgical procedures. In some embodiments, the present invention provides a screening platform for selecting compounds such as small molecules that can be tested for neuroprotection therapies. In some embodiments, the present invention can be used to screen for molecules with potential therapeutic effects in patients with retinal degenerative diseases. In other embodiments, the present invention can be used to generate human retinal tissue for cell therapy or tissue transplantation to treat patients with retinal diseases.

The screening methods of the present invention are not limited to the specific type of the compound. Potential test compounds include chemical agents (such as toxins), pharmaceuticals, peptides, proteins (such as antibodies, cytokines, enzymes, etc.), and nucleic acids, including gene medicines and introduced genes, which may encode therapeutic agents such as proteins, antisense agents (i.e. nucleic acids comprising a sequence complementary to a target RNA expressed in a target cell type, such as RNAi or siRNA), ribozymes, etc. Additionally or alternatively, the assays may screen for a physical agent such as radiation (e.g. ionizing radiation, UV-light or heat); these can be tested alone or in combination with chemical and other agents. In one embodiment, entire compound libraries are screened. Compound libraries are a large collection of stored compounds utilized for high throughput screening. Compounds in a compound library can have no relation to one another, or alternatively have a common characteristic. For example, a hypothetical compound library may contain all known compounds known to bind to a specific binding region.

The assays may also be used to test delivery vehicles. These may be of any form, from conventional pharmaceutical formulations to gene delivery vehicles. For example, the assays may be used to compare the effects of the same compound administered by two or more different delivery systems (e.g. a depot formulation and a controlled release formulation). They may also be used to investigate whether a particular vehicle could have effects by itself. As the use of gene-based therapeutics increases, the safety issues associated with the various possible delivery systems become increasingly important. Thus the models of the present invention may be used to investigate the properties of delivery systems for nucleic acid therapeutics, such as naked DNA or RNA, viral vectors (e.g. retroviral or adenoviral vectors), liposomes, etc. Thus the test compound may be a delivery vehicle of any appropriate type with or without any associated therapeutic agent. Non-limiting examples of delivery vehicles include polymersomes, vesicles, micelles, plasmid vectors, viral vectors, and the like.

### Tissue Engineering

In some embodiments, the scaffolds of the present invention can be used to replace or regenerate tissue to treat defects and wounds. Eye-related defects include but are not limited to macular degeneration, retinal detachment, uveitis, glaucoma, retinitis, and color blindness. Wounds for which the scaffolds are useful in promoting closure include, but are not limited to, abrasions, avulsions, contusions, incised wounds, open wounds, penetrating wounds, perforating wounds, puncture wounds, surgical wounds, subcutaneous wounds, or tangential wounds. In some embodiments, the scaffolds promote differentiation to regenerate the various substructures of the eye, including but not limited to the choroid, pigment epithelium, photoreceptors, horizontal cells, bipolar cells, amacrine cells, ganglion cells, inner and outer plexiform layers, and the like. The scaffolds may be secured to treatment area using sutures or adhesives. The scaffolds may be cut to match the size of a treatment area, or may overlap the edges of a treatment area.

In some embodiments, the scaffolds are applied cell-free, such that upon implantation, the scaffolds support cell migration and proliferation from native tissue. The cell-free scaffolds can be supplemented with ECM and other cellular secretions to promote healing. In other embodiments, the scaffolds are seeded with one or more populations of cells to form an artificial tissue construct. The artificial tissue construct may be autologous, where the cell populations are derived from a subject's own tissue, or allogenic, where the cell populations are derived from another subject within the same species as the subject. The artificial organ construct may also be xenogenic, where the different cell populations are derived from a species that is different from the subject. For example the cells may be derived from organs of mammals such as humans, monkeys, dogs, cats, mice, rats, cows, horses, pigs, goats, and sheep.

In some embodiments, the present invention provides a method for treating an eye-related disorder or defect comprising implanting the scaffold described herein into an eye of a subject in need thereof. For example, in some embodiments, the method comprises generating a scaffold, seeding the scaffold with one or more cell populations as described herein, and implanting the scaffold into the eye of a subject in order to treat the eye-related disorder or defect. In certain embodiments, the method comprises culturing the scaffold ex-vivo to promote the differentiation of the cell populations prior to implantation.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out embodiments of the present invention, and are not to be construed as limiting in any way the remainder of the disclosure.

### Example 1

### A Biodegradable Scaffold Enhances Differentiation and Enables Retinal Progenitor Cells to form a Planar Sheet

The compositions, methods and results presented herein, combine the best features of the retinal organoids and scaffolds. hESCs were differentiated into RPCs on a scaffold composed of gelatin, chondroitin sulfate, and hyaluronic acid (GCH), naturally occurring components of the retinal extracellular matrix (J. Kundu, et al, Acta Biomater., 2016, 31: 61-70). The cultures were suitable for forming a planar, laminated neo-retina and delivering partially differentiated RPE into the subretinal space of a mouse model of retinal degeneration. Further information regarding the data presented herein can be found in Singh et al. (2018, Biomaterials, 154: 158-168), which is incorporated by reference herein in its entirety.

### Materials

Gelatin type A from fish skin was purchased from J. T. Baker (Phillipsburg, N.J.). Chondroitin sulfate, >90% was obtained from Alfa Aesar, Ward Hill, Mass. (USA), hyaluronic acid from Calbiochem/Millipore (San Diego, Calif.), and ammonium persulfate from Fisher Scientifics (Fair Lawn, N.J.). 1-Ethyl-3-(3-dimethylaminopropyl)-carbodiimide-N-hydroxysuccinimide (EDC-NHS), Tri-Buffer-saline with 1% Tween 20, Phosphate buffer saline (PBS) (pH 7.4, 1.4M NaCl, 0.1M phosphate, 0.03M KCL) were all purchased from American BIO (Natick, Mass.). Glutaraldehyde, 50% was obtained from Merck (Solon, Ohio). Milli-Q-grade water was used in all experiments except for PCR in which nuclease free-water from the Bio-Rad i-Script cDNA synthesis kit was used. iTaq® Universal SYBRGreen Supermix, and custom PCR arrays were manufactured and validated by Bio-Rad (Hercules, Calif.). AlamarBlue was obtained from Accurate Chem (Waterbury, N.Y.). Unless indicated otherwise, all other chemicals and solvents, used without further purification, were purchased from Sigma-Aldrich (St. Louis, Mo.).

### Methods

Preparation of 3D Scaffold

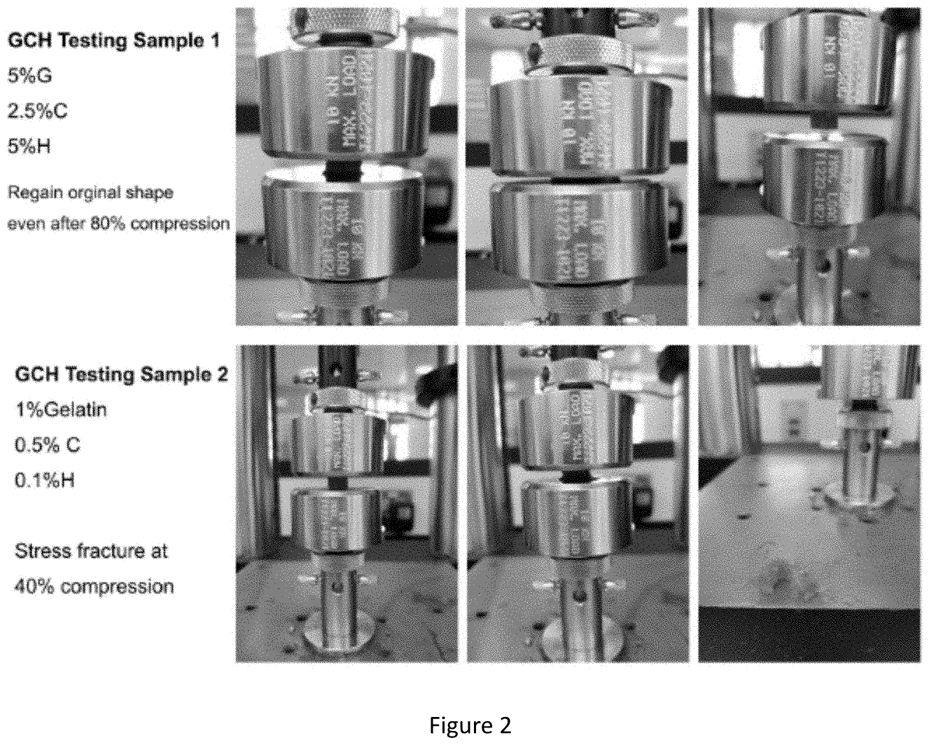

Scaffolds were prepared with degassed, double-distilled water. Different combinations of polymers and cross-linkers were used to fabricate the GCH scaffold (Table 1). The cross-linkers were glutaraldehyde or EDC-NHS. The solution was frozen in a 10 ml syringe at −80° or −20° C., as indicated in Table 1, for 18 hrs. The preparation was removed from the syringe and vacuum dried in a lyophilizer to yield a solid 3D scaffold block. To estimate compressive strength, scaffolds were hydrated in PBS overnight and mounted in an Instron 5967 Tensile & Compression Tester, (Norwood, Mass.). Scaffolds were compressed to 60% of their original height at the rate of 10 kN/min and scaffolds that did not fracture were further used for experimentation (FIG. 2). Scaffolds that were mechanically stable were frozen in OCT and sectioned using a cryotome to create 60 μm thick planar sheets.

TABLE 1
COMPOSITION AND SYNTHESIS
CONDITIONS FOR GCH SCAFFOLD

| | Synthesis Conditions | | | | | |
|---|---|---|---|---|---|---|
| | A | B | | C | D | E |
| Gelatin¹ | 4 | 5 | | 0.5 | 1 | 0.5 |
| Chondroitin | 2.5 | 2.5 | | 0.5 | 1 | 0.5 |
| Sulfate¹ | | | | | | |
| Hyaluronic | 5 | 5 | | 1 | 1 | 1 |
| Acid¹ | | | | | | |
| Cross linker | GA² | GA | | GA | EDC-NHS³ | EDC-NHS |
| Cross-linker | 100 μl | 50 | μl | 50 μl | 100 μl | 50 μl |
| volume | | | | | | |
| Incubation | −80 | −20 | | −20 | −20 | −20 |
| temperature | | | | | | |
| (° C.) | | | | | | |
| Incubation | 20 | 18 | | 16 | 14 | 20 |
| time (hr) | | | | | | |
| Compressive | Failed | 5 | MPa | Stress | Dissolves | Dissolves |
| strength | at 40% | | | fracture | when | at room |
| (Mpa⁴) | strain | | | at 25% | thawed | temperature |
| | | | | strain | | |

¹w/v %
²Glutaraldehyde
³1-Ethyl-3-(3-dimethylaminopropyl)-carbodiimide-N-hydroxysuccinimide (EDC-NHS)
⁴Megapascal

Human Embryonic Stem Cells (WA09) Culture and Differentiation

Undifferentiated WA09 human embryonic stem cells (hESC) were cultured on 1% Matrigel coated plates and maintained in mTeSR-1 media. Media was changed every 2 days and colonies were regularly scraped under a sterile microscope to remove spontaneously differentiating cells. For passaging, colonies were lifted by incubating with 1 U/ml dispase for 30 min at 37° C., then triturated and washed with DMEM/F-12 nutrient media and plated onto 1% Matrigel-coated dishes.

WA09 cells were differentiated using a published protocol (Eiraku M, et al. Nature, 2011, 472:51-56). Briefly, cells were treated with blebbistatin, dissociated with dispase incubation, and cultured in low-attachment, Lipidure-COAT Plates (Amsbio, Abingdon, UK) to generate embryoid bodies of uniform size. On day 0 (D0), embryoid bodies were incubated in mTeSR-1 medium (Stemcell technologies, Vancouver, BC). Neural induction medium (NIM) contained Dulbecco's modified Eagle's medium (DMEM) high glucose and F-12 nutrient medium (Gibco/Life Technologies, Grand Island, N.Y.) with 15% knockout serum (Invitrogen/Thermo-Fischer, Waltham, Mass.), 1% N2 supplement, 0.1 mM 2-mercaptoethanol (Sigma-Aldrich), 0.1M nonessential amino acids (Invitrogen), 1 mM glutamax, 50 U/ml penicillin, 50 μg/ml streptomycin, and 2 μg/ml heparin. NIM was use was diluted with mTeSR-1 as follows: D1, 1:3 (NIM:mTeSR-1); D2, 1:1; and D-D7, undiluted NIM. Then the suspensions of embryoid bodies were seeded on Matrigel coated 6-well plates or on the scaffold (10 embryoid bodies per well or scaffold) for further experimentation. Before seeding, scaffolds were sterilized overnight with 70% ethanol, washed three times with PBS, treated with pen-strep for 30 mins and finally left in NIM overnight.

After 21 days, when retinal cups appeared in the Matrigel cultures, the retinal cups were isolated and maintained in suspension culture using a serum free medium that contained DMEM-F12 (3:1) along with 2% B27 (Gibco/Life Technologies) and 50 U/ml penicillin, 50 m/ml streptomycin. At D21 cells cultured on the scaffold were also switched to this medium. Medium was changed 2 to 3 times a week for the remainder of the experiment.

Proliferation Assay

Ki-67 is a marker for dividing cells that is expressed in the nucleus during interphase, but is absent in quiescent cells. DAPI (4′,6-Diamidino-2-Phenylindole, Dihydrochloride; Life technologies, Eugene, Oreg.) was used to identify total nuclei. The ratio of Ki-65 positive cells/total nuclei was estimated by Image J (https://imagej.nih.gov/ij/) to count cells in three random fields (>1000 cells/field) from three sets of experiments.

Quantitative Real-Time RT-PCR (qRT2-PCR)

Total RNA was extracted using RNeasy mini kit (Qiagen). cDNA was reverse transcribed using 2 μg of total RNA using QuantiTect Reverse Transcription kit (BioRad). Select genes were analyzed using iTaqSYBR Green (BioRad) and RNA primers synthesized at Keck Center (Yale University) (listed in Table 2). Samples were further tested using customized PCR array for 48 genes specific for early eye field, neuroretinal, and RPE markers. Relative mRNA expression was normalized with housekeeping genes (GAPDH and Actin) and calculated using the 2-ΔΔCt method (K. J. Livak, T. D. Schmittgen, Methods, 2001, 25(4):402-8).

TABLE 2
List of primers

| Gene | Cell type | Sequence |
|---|---|---|
| RCVRN | Photoreceptor | 5′-CCA GAG CAT CTA CGC CAA GT-3′ (SEQ |
| | | ID NO: 1) |
| | | 3′-CAC GTC GTA GAG GGA GAA GG-5′ (SEQ |
| | | ID NO: 2) |
| OTX2 | Early eye field | 5′-GCA GAG GTC CTA TCC CAT GA (SEQ ID |
| | | NO: 3) |
| | | 3′-CTG GGT GGA AAG AGA GAA GC TG-5′ |
| | | (SEQ ID NO: 4) |
| CHX10 | Retinal | 5′-ATT CAA CGA AGC CCA CTA CCC AGA- |
| | progenitor | S′ (SEQ ID NO: 5) |
| | cells/Bipolar | 3′-ATC CTT GGC TGA CTT GAG GAT GGA- |
| | cells | 5′ (SEQ ID NO: 6) |
| GAPDH | Housekeeping | 5′-TCA CCA GGG CTG CTT TTA AC-3′ (SEQ |
| | gene | ID NO: 7) |
| | | 3′-GCA AAG CTT CCC GTT CTC AG-5′ (SEQ |
| | | ID NO: 8) |
| LHX2 | LIM Homeobox | 5′-TAG CAT CTA CTG CAA GGA AGA C-3′ |
| | Protein 2 for | (SEQ ID NO: 9) |
| | neural cells | 3′-GTG ATA AAC CAA GTC CCG AG-5′ (SEQ |
| | | ID NO: 10) |
| NANOG | ES cell | 5′-CAA AGG CAA ACA ACC CAC TT-3′ (SEQ |
| | proliferation, | ID NO: 11) |
| | renewal, and | 3′-TCT GCT GGA GGC TGA GGT AT-5′ (SEQ |
| | pluripotency | ID NO: 12) |
| Oct-4 | Stem cells | 5′-CGA GCA ATT TGC CAA GCT CCT GAA- |
| | pluripotency | 3′ (SEQ ID NO: 13) |
| | marker | 3′-TTC GGG CAC TGC AGG AAC AAA TTC-5′ |
| | | (SEQ ID NO: 14) |
| PAX6 | Neural retinal | 5′-TCT AAT CGA AGG GCC AAA TG-3′ (SEQ |
| | development | ID NO: 15) |
| | | 3′-TGT GAG GGC TGT GTC TGT TC-5′ (SEQ |
| | | ID NO: 16) |
| RAX | Retina and | 5′-GAA TCT CGA AAT CTC AGC CC-3′ (SEQ |
| | Anterior Neural | ID NO: 17) |
| | Fold Homeobox | 3′-CTT CAC TAA TTT GCT CAG GAC-5′ |
| | | (SEQ ID NO: 18) |
| SIX3 | Neural | 5′-GGA ATG TGA TGT ATG ATA GCC-3′ |
| | progenitor | (SEQ ID NO: 19) |
| | cells | 3′-TGA TTT CGG TTT GTT CTG G-5′ (SEQ |
| | | ID NO: 20) |
| SOXI | Anterior | 5′-GGA CT A GTT GAA TGT AC A GCA TGA |
| | forebrain | TGG A-3′ (SEQ ID NO: 21) |
| | | 3′-CGG AAT TCG ATG TGT GTC AGT GGC |
| | | ATG GT-5′ (SEQ ID NO: 22) |

Immunobiochemistry

At different time points, protein was extracted from WA09-GCH cultured using a lysis buffer composed of complete™ Protease Inhibitor Cocktail (Sigma-Aldrich, 1.0% NP-40, 150 mM NaCl, 0.5% sodium deoxycholate, 0.1% SDS, and 50 mM Tris, pH 8.0. Samples from D21, D31, and D51 were immunoblotted for LHX2, PAX6, OTX2, recoverin, and rhodopsin. Blots were imaged using Image J software and normalized to actin.

### Immunofluorescence Confocal Microscopy

Every week, cultures were examined for cell migration and differentiation. Cultures and tissues were fixed in 4% paraformaldehyde for 5 mins, washed, and incubated with graded sucrose solutions until a concentration of 30% was achieved. The samples were then incubated overnight at 4° C. in a 1:1 mixture of OCT (Fisher Healthcare™, Pittsburg, Pa.) and 30% sucrose. Sections (12 μm) were cut using a Leica CM1950 cryostat (Buffalo Grove, Ill.) at −23° C. mounted on poly-lysine coated slides and dried for 48 hrs before immunocytochemistry. Slides were washed with cold PBS, permeabilized with 0.1% Triton-X100 in PBS, and blocking with PBS containing 10% donkey serum and 0.1% Triton-X100. The sections were incubated overnight with primary antibodies (listed in Table 3). Because some of the antibodies were mouse monoclonals, they exhibited background fluorescence in the choroid and retinal blood vessels. Because the retina is an immune-privileged space, background fluorescence in the retina was below the signal for the targeted antigen. The slides were washed three times with PBS before incubation with secondary antibodies conjugated with Cy2, Cy3, or Cy5 (Jackson ImmunoResearch Laboratories, West Grove, PA). DAPI (4,6-diamidino-2-phenylindole) was used to label the nucleus. Slides were washed 3 more times 3× with PBS. Fluorescence images were captured with an LSM 410 spinning-disc confocal microscope and processed using Zen software (Carl Zeiss, Inc, Thornwood, N.Y.). Images used are representative of 3 or more experiments.

TABLE 3
Details of Primary antibodies

| Target | Antigen | Host1 | Dilution2 | Supplier |
|---|---|---|---|---|
| Anterior | Sox1 | RP | IF 1:400 | Abcam |
| forebrain | | | | |
| Mitotic marker | Ki67 | MP | IF 1:200 | Invitrogen |
| Early Eye Field | PAX6 | RM | IF 1:100 | Abgent |
| | RAX | RP | IB 1:5000 | Novus Biologicals |
| | LHX2 | GP | IF 1:200 | Santa Cruz Bio |
| | CHX10 | SP | IF 1:300 | EMD Millipore |
| | OTX2 | MM | IB 1:5000 | Novus Biologicals |
| | | | IF 1:300 | |
| | | | IF 1:200 | |
| | | | IB 1:3000 | |
| | Recoverin | RP | IF 1:400 | EMD Millipore |
| | | | IB 1:5000 | |
| Photoreceptor | CRX | GP | IF 1:300 | Fisher Scientific |
| Rods/Cones | Rhodopsin | MP | IF 1:300 | Cell Signaling |
| | | | IB 1:3000 | Technology |
| Neural retinal | Prox1 | RM | IF 1:500 | Fisher Scientific |
| cells precursor | β-tubulin III | MP | IF 1:200 | Fisher Scientific |
| Human antigen | TRA-1-85 | MM | IF 1:300 | EMD Millipore |
| | Ku80 | MM | IF 1:250 | Novus |
| Inflammatory | IL-6 | MM | IF 1:200 | Abcam |
| cells | | | | |
| Microglia | IBA-1 | GM | IF 1:200 | Abcam |
| Normalization | Actin | MP | IB 1:5000 | Sigma |

1RP, Rabbit Polyclonal; RM, Rabbit Monoclonal; MM, Mouse Monoclonal; MP, Mouse Polyclonal; SP, Sheep Polyclonal; GP, Goat Polyclonal
2IF, Immunofluorescence; IB, Immunoblot

Tissue Graft in RD10 Mice:

On postnatal day 30 (P30) when the outer nuclear layer (ONL) was ≥75% degenerated (C. Gargini, et al, J. Comp. Neurol. 2007, 500(2): 222-238). RD-10 mice were anesthetized by intramuscular injection of a mixture of ketamine (100 mg/kg) and xylazine (10 mg/kg). A small scleral hole was made after conjunctival incision, and a local retinal detachment was induced by injecting PBS (1 μl) into the subretinal space. In some experiments, the PBS contained a suspension of 50,000 dissociated cells. The scleral incision was enlarged to insert D21 cultures (14 days post-seeding on the scaffold) into the dorsal quadrant with the cellular (photoreceptor precursor) side facing the neurosensory retina. The scaffold (0.5×0.5 cm) was inserted using a Dumont #5 forceps. Animals were divided into four different groups (three animals/group) that received: 1) no surgery, 2) a cell suspension of dissociated retinal eyecups that had been isolated on D21 from Matrigel-cultured cells, 3) an implant of GCH scaffold without cells, and 4) an implant of a GCH-WA09 culture at D21. D21 cultures were selected, because retinal progenitor cells were present, but differentiation was not advanced and could be influenced by both the scaffold and interactions with the host. After transplantation, the mice were transferred into a dark room for 1-2 days, and then maintained in a regular animal facility. Immunosuppression was not used. The animals were euthanized at 3, 6, or 12 weeks post-surgery, and the eyes harvested to process the tissues for immunofluorescence.

Statistical Analysis

All data presented in this manuscript is shown as the mean±standard deviation (SD) unless otherwise indicated. All experiments presented here were completed in biological and technical triplicates. The data from the experimental sets were compared with controls and statistical analyses was performed by one-way ANOVA, and p values <0.05 were considered statistically significant.

### Results

Properties of the GCH Scaffold

Three polymers were selected based on their biological properties. Collagen, chondroitin sulfate, and hyaluronic acid are natural components of the retinal extracellular matrix (J. Kundu, et al, Acta Biomater., 2016, 31: 61-70). For collagen, gelatin was substituted, denatured collagen that is non-immunogenic and promotes cell attachment (J. Zhu, et al, Expert Rev. Med. Devices, 2011, 8(5): 607-626). Chondroitin sulfate promotes the differentiation of stem cells (A. Purushothaman, et al, J. Biol. Chem., 2012, 287(5): 2935-2942). Hyaluronic acid is a retinal growth factor (M. Inatani, et al, Prog. Retin. Eye Res., 2002, 21(5): 429-447). The various combinations of these polymers and cross-linking agents are shown in Table 1. Scaffolds were discarded if they were too soft to form a stable matrix or insufficiently cross-linked to prevent melting in buffer solution. EDC-NHS proved to be an unsatisfactory cross-linker. Substituting glutaraldehyde as the cross-linker, the concentrations of glutaraldehyde and ratios of the polymers were varied, until a satisfactory scaffold was identified (Table 1, Column “B”). The final optimal concentrations were 5% gelatin, 2.5% chondroitin sulfate and 5% hyaluronic acid. At this concentration, the scaffold was stable and spongy with a compressive strength of 5.0±0.2 MPa (FIG. 2). Scanning electron and bright-field microscopy revealed that the scaffold was a homogenous, interconnected network of pores with diameters that ranged from 150-190 μm in diameter (FIG. 3A and FIG. 3B).

Cell Attachment and Proliferation on the GCH scaffold

After seven days of differentiation, an equal number of embryoid bodies were seeded on the GCH scaffold or on Matrigel coated plates. Twenty-four hours after seeding the embryoid bodies were firmly attached to scaffold, as confirmed by bright-field and scanning electron microscopy (FIG. 3C and FIG. 3D). By D14 (7 days post-seeding on the scaffold), cell nuclei stained with DAPI revealed the cells penetrated the depth of the 60 μm thick scaffold (FIG. 3E) By D28, cells had penetrated the depth of a 140 μm thick scaffold in some places (FIG. 3F). The 60 μm scaffold was used for all further experiments.

Proliferation of the cells in control (Matrigel) and GCH cultures was monitored using Ki67. By D14, the percentage of proliferating cells decreased in the GCH cultures relative to the control (FIG. 4A). By D31, the percentage of proliferating cells was the same in each culture. Apoptosis was not evident: Caspases were not expressed and a caspase substrate, poly (ADP-ribose) polymerase-1 (PARP-1), was not cleaved (FIG. 4B). A decrease in proliferation has been correlated with an increase in cell differentiation (M. M. Estefania, et al, Sci. Rep. 2012, 2: 279).

Differentiation Into RPC was Enhanced on the GCH Scaffold

By D3, cells on the scaffold decreased the expression of pluripotency markers and began to increase the expression of LHX2, an early eye field marker, as determined by qRT²-PCR (FIG. 4C). SOX1, a marker for non-retinal, anterior forebrain cells, was evident on D7, but not thereafter. The expression of early eye field genes and recoverin were compared between scaffold and Matrigel cultures on D24 (FIG. 4D). Normalized SIX3 expression was the same for both cultures, but the expression of the other genes was significantly higher in the scaffold cultures. A qRT²-PCR microarray was used to assess the expression of a variety of markers for the various retinal cell types. On D35, the great majority of the markers were expressed >4× that of Matrigel cultures (FIG. 4E). Note that by this time, retinal eyecups were isolated from undifferentiated and anterior forebrain cells of the Matrigel cultures. In contrast, the scaffold cultures lacked a similar purification step, which makes the increased expression relative to the normalizations genes all the more impressive. Increases were found for eye field transcription factors (RAX, SIX3 and OTX2), photoreceptor genes (CRX, GNAT1, NRL, and RHO), interneuron genes (PROX1, CALB2) and ganglion cells (POU4F1, POUF4F2, TUBB2). For the proteins tested, these findings were confirmed by immunoblotting (FIG. 4F). Notably, rhodopsin could be detected as early as D51, much earlier than D90-115, as reported in the literature (Reichman, S, P Natl Acad Sci USA, 2014. 111:8518-8523; Zhong, X, Nat. Commun, 2014, 5:4047; K. J. Wahlin, K J, et al., Sci. Rep., 2017, 7:766.). Similar results were obtained using human induced pluripotent cells as the source of the stem cells (FIG. 5A-FIG. 5C).

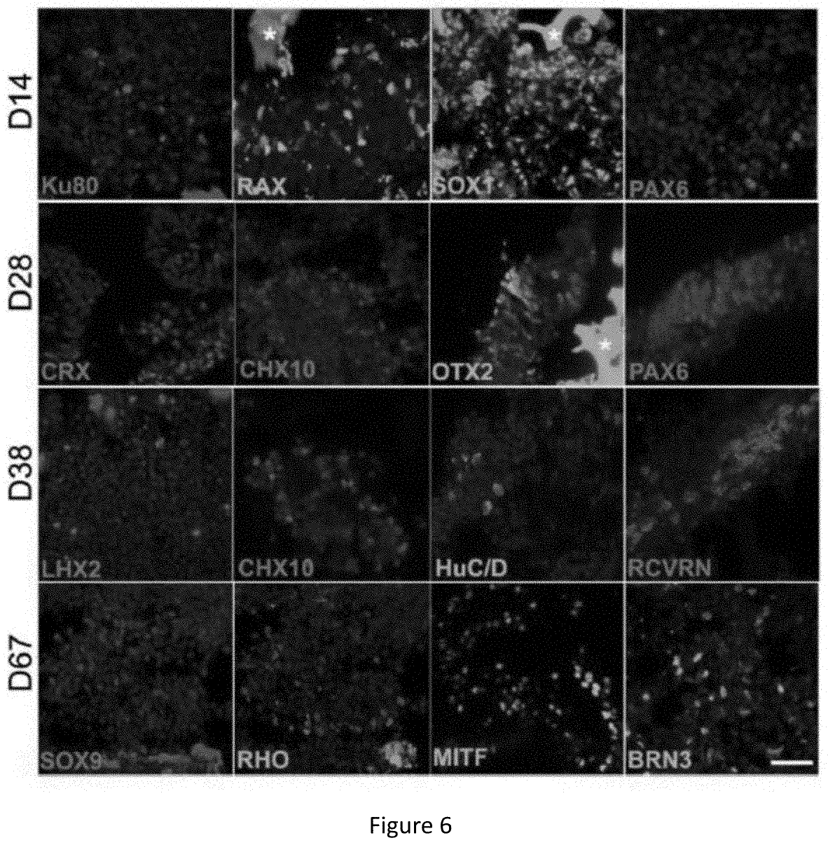

Immunocytochemistry confirmed that the anterior forebrain marker, SOX1, was expressed on D14 along with early eye field markers PAX6 and RAX (FIG. 6). By D28, additional RPC markers were evident, including: CRX, CHX10, and OTX2. CRX is an early marker for rod and cone photoreceptors and plays critical role in RPC differentiation (T. Furukawa, et al, Cell, 1997, 91(4): 531-541). CHX10 (VSX2) is a transcriptional factor that favors proliferation of early RPC and promotes the differentiation of bipolar cells by affecting the differentiation of late progenitor cells (S. Rowan, et al, Dev. Biol., 2004, 271(2):388-402; N. S. Dhomen, et al, Invest. Ophthalmol. Vis. Sci., 2006, 47(1): 386-396). OTX2 is a homeobox gene that directs cells to a photoreceptor cell fate (A. Nishida, et al, Nat. Neurosci., 2003, 6(12):1255-1263). By D31, LHX2, CHX10, HuC/D (ganglion cells) and recoverin (rod photoreceptors) were expressed. By D67, SOX9 (Muller glia), rhodopsin (rod photoreceptors), MITF (retinal pigmented epithelium marker) and BRN3 (POU4F1, POU4F2; ganglion cell markers) were evident.

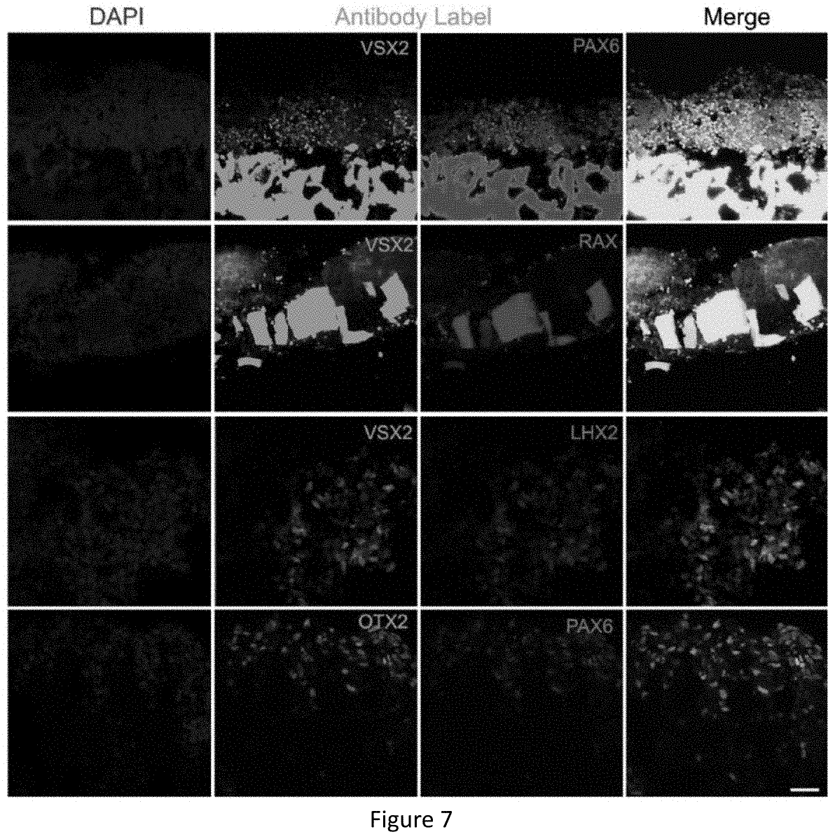

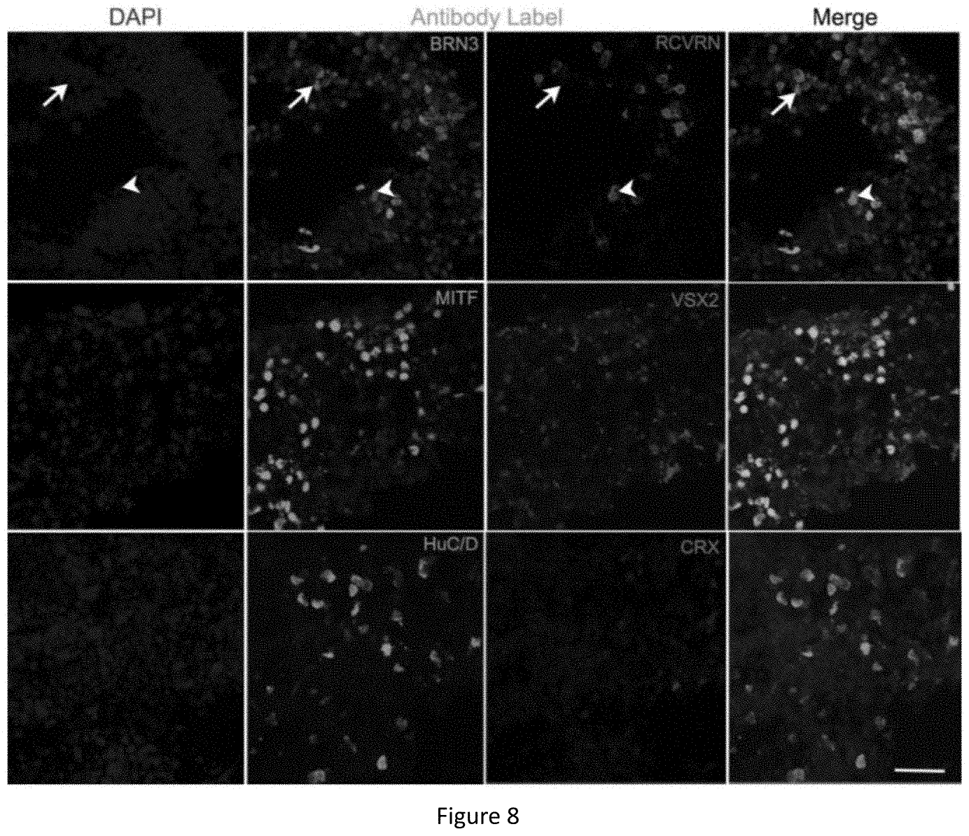

Double immunofluorescence labeling was used to further characterize differentiation. On D31, a thick layer of cells was identified adjacent to the scaffold that was enriched in VSX2⁺ and PAX6⁺ cells (FIG. 7). The vast majority the PAX6⁺ cells were co-labeled with VSX2 (specification and morphogenesis of the neurosensory retina), but only a subset of VSX2⁺ cells were co-labeled with PAX6. PAX6 initially labels all RPC, but is later restricted to inner retinal layers. The vast majority of VSX2⁺ cells, co-labeled with other retinal markers, such as RAX (specification of the neurosensory retina) and LHX2 (early neuronal development). This finding suggests most of the neuronal cell types are precursors for neurosensory retina. The vast majority of PAX6⁺ cells, co-labeled with OTX2, which marks photoreceptor cells when found in the retina. A large number of unlabeled cells (DAPI only) were also observed. On D90, cells from different retinal layers were observed in the same microscopic field, but they were not segregated into distinct domains or lamina (FIG. 8). There was little co-labeling between MITF and VSX2. At this stage of differentiation MITF should label RPE, while VSX2 would label primarily bipolar cells. CRX, a photoreceptor marker, did not co-label HuC/D⁺ cells (a ganglion cell marker), and a second photoreceptor marker, RCVRN, did not co-label BRN3+ cells (a second ganglion cell marker). These data indicate that different retinal cell types were in evidence, but that cells only partially segregated into distinct domains.

Engraftment of RPC into RD10 Mice Using the GCH Scaffold

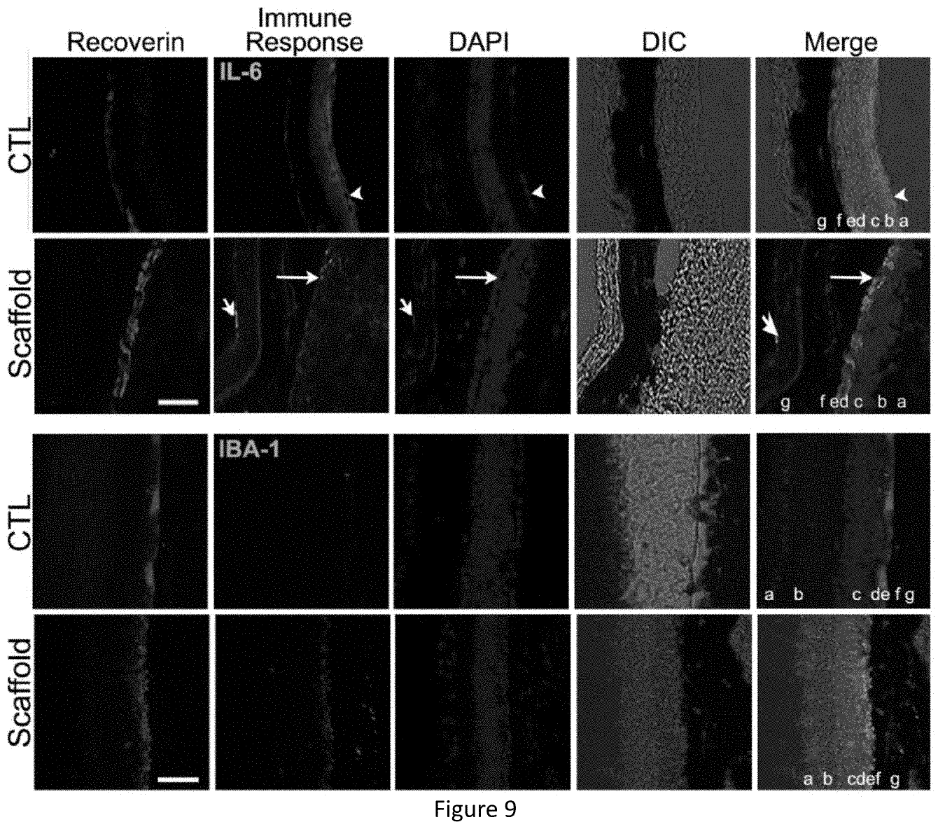

The biocompatibility of the GCH scaffold was tested by implanting it into the sub-retinal space of P30, rd10 mice. Three weeks after implantation (P51), evidence of IL-6 was minimal at the site of implantation (FIG. 9). In the controls, IL-6 immunoreactivity was observed near Bruch's membrane, the interface of the choroid and RPE, but not in the subretinal space. Immunoreactivity was also observed in large blood vessels that were also seen in the controls. The immunoreactivity of the controls likely reflects background staining from use of a mouse monoclonal antibody on mouse tissue that was outside the immune-privileged space of the retina. In the test eyes, the scaffold had degraded by this time without overt effects on the histology of the retina. Recoverin immunoreactivity revealed the host photoreceptor layer and the extent of the retinal degeneration. As expected, only one row of photoreceptor nuclei was evident in the ONL of the controls, and this was also observed at the site of the implant. Only a few cells were observed with intense immunoreactivity for IL-6. Ionized calcium-binding adapter molecule-1 (IBA-1) was also used to search for activated microglial cells (FIG. 9). Microglia are the major resident immune cells in the central nervous system and the retina (W. Ma, et al, Plos One, 2009, 4(11):e7945). In the retina, microglial proliferation and activation occur during local injury. At 6 weeks post-implantation, there was evidence of a few microglial cells at the transplant site compared to non-implanted retina. RPC-GCH appeared to be well-tolerated in the sub-retinal space and did not trigger a major immune reaction in the rd10 mice.

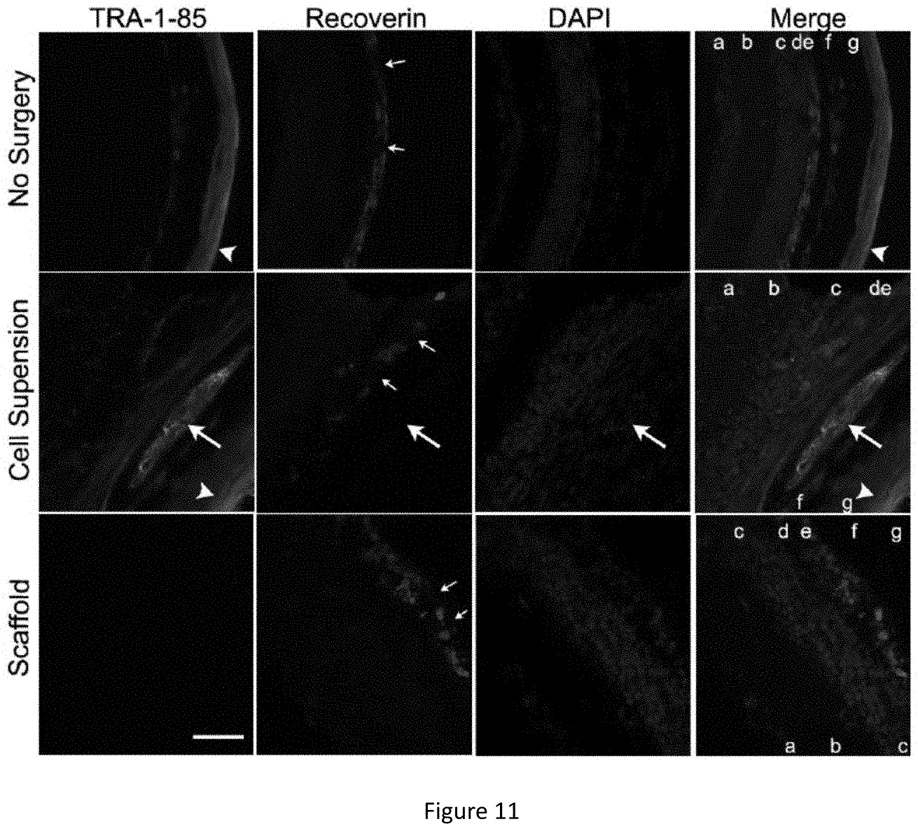

Scaffolds seeded with WA09 hESC (D21) were implanted into the subretinal space of P30, rd10 mice. Implanted cells were distinguished from host cells by two human antigens, TRA-1-85 and Ku-80. Because TRA-1-85 is a membrane antigen, the fluorescent signal associated with its label would be accentuated where membranes were concentrated, such as apical microvilli. The Ku-80 antibody that was used labels human double stranded DNA in both the nucleus and mitochondria. Its fluorescent signal would be most concentrated in the cell bodies and in puncta along neuronal processes. Two weeks post-implantation (P44), the ONL, revealed by recoverin immunoreactivity, was 2-3 rows thicker than the age-matched control (FIG. 10). Intense TRA-1-85 immunoreactivity was observed in cells lining the subretinal space. TRA-1-85 was observed with recoverin in those cell bodies, but not the presumptive, apical microvilli. Recoverin⁺ cells not in contact with the subretinal space did not label with TRA-1-85, indicating that these were host photoreceptor cells that had been preserved. This transient preservation of host cells was not observed in cell-only or scaffold-only controls (FIG. 11). Note that in FIG. 9, scaffold without cells failed to preserve the ONL.

Six to eight weeks post-implantation (P72 and P84), there was one continuous row of cells that were positive for recoverin and Ku-80 (FIG. 10). Notably, the nuclei of the double-labeled cells were not labeled by Ku-80. Instead, both labels identified the cytoplasm, where recoverin and mitochondria would be found. Besides double-labeled cells, Ku-80-positive cells were also found in the inner plexiform layer near the retinal ganglion cell layer. Ku-80 did label the nuclei of those cells along with clusters of mitochondria that lie like beads-on-a-string along neuronal-like processes. These processes ran laterally along the inner plexiform layer and into the outer plexiform layer. Cells survived as long as 12 weeks post-transplantation, but some variability was observed. The cells lining the ONL were not always positive for recoverin. An example is shown in FIG. 10, P84.

### Example 2

### Laminin 521 Promotes the Formation of a Planar Retinal Organoid

A scaffold that supports the differentiation of planar retinoids and rapidly degrades when implanted into the subretinal space is described herein. The scaffold is composed of chondroitin sulfate, collagen, and hyaluronic acid, which are naturally occurring components of the retinal extracellular matrix (Kundu et al. 2016). It has been demonstrated that this combination provides a niche that favored retinal differentiation over other cells of the anterior forebrain, but did not provide a uniform planar retinoid. To improve uniformity by increasing cell attachment, laminin 521 was tested, a major component of the retina's inner limiting membrane (Balasubramani et al. 2010; Pinzón-Duarte et al. 2010). Laminin 521 is also found in stem cell niches, where it promotes cell proliferation (Laperle et al. 2015; Polisetti et al. 2017). Because the retinoid is planar, the effects of co-culturing it with the RPE and its ability to implant RPC into a mouse model of retinal degeneration were tested.

### Materials and Methods

Materials

Chondroitin sulfate, >90%, was obtained from Alfa Aesar, Ward Hill, Mass. (USA), gelatin type A from fish skin from J. T.Baker (Phillipsburg, N.J.), and hyaluronic acid from Calbiochem/Millipore (San Diego, Calif.). Ammonium persulfate was obtained from Fisher Scientifics (Fair Lawn, NJ) and glutaraldehyde, 50%, from Merck (Solon, OH). Tri-Buffer-saline with 1% Tween 20 and phosphate buffer saline (PBS) (pH 7.4, 1.4M NaCl, 0.1M phosphate, 0.03M KCL) were purchased from American BIO (Natick, Mass.). Milli-Q-grade water was used in all experiments except for gene expression in which nuclease free-water from Bio-Rad i-Script cDNA synthesis kit was used. iTaq® Universal SYBRGreen Supermix, and custom PCR arrays were manufactured by Bio-Rad (Hercules, Calif.). AlamarBlue was obtained from Accurate Chem (Waterbury, N.Y.).

Unless indicated otherwise, all other chemicals and solvents, used without further purification, were purchased from Sigma-Aldrich (St. Louis, Mo.).

Scaffold Fabrication with Laminin-521

Gelatin (500 mg), chondritin sulfate (250 mg) and hyaluronic acid (500 mg) was dissolved in 10 ml double-distilled, degassed water. To cross-link the scaffold, glutaraldehyde, 0.5%, was added, the solution was frozen in a 5-ml syringe barrel at −20° C. for 16-18 hrs, and vacuum dried in a lyophilizer. The 3D scaffold monolith was cut into 8 mm thick blocks, embedded into OCT and sectioned to create 60 μm thick, 1.0 cm-diameter, planar sheets. The planar sheets were sterilized by incubating them in a series of graded alcohols (20% to 80%) and stored at 4° C. On the day of the experiment, scaffolds were placed in a 48 well plate washed three times with PBS, treated with penicillin-streptomycin for 30 mins and finally left in NIM overnight at 37° C. in a humidified incubator. The NIM was removed and the scaffolds dried by incubating them at 37° C. Laminin-521 (Stem cell technologies, BC, CA) was diluted in media to 1.0 μg/ml and 20 μl was dropped onto each scaffold after the NIM had been removed. These scaffolds were incubated a 37° C. for 30 mins to allow complete absorption of the lamini-521. The GCH-521 scaffolds were seeded with RPC after a 30-min incubation.

Retinal Progenitor Cells Generation from hESCs:

Undifferentiated WA09 cells were maintained and differentiated as described previously (Singh et al., 2017). Briefly, RPC were differentiated by dissociating WA09 with 1 U/ml dispase, suspended in mTeSR-1 (Stemcell technologies, BC, CA) that included 10 μm blebbstatin and 5 mM rock inhibitor containing and cultured in low-attachment six well plates (Corning, N.Y., USA) to generate embryoid bodies at day 0 (D0). The next day, mTeSR-1 was gradually exchanged for neural induction medium (NIM) medium: 50% Dulbecco's modified Eagle's medium, high glucose (DMEM)/50% F-12 nutrient medium (Gibco/Life technologies, Grand Island, N.Y.) supplemented with 15% knockout serum (Invitrogen/Thermo-Fischer, Waltham, Mass.), 0.1 mM 2-mercaptoethanol (Sigma-Aldrich), 1% N2 supplement, 1 mM glutamax, 0.1 M nonessential amino acids (Invitrogen), 50 U/ml penicillin, and 50 μg/ml streptomycin. The ratio of mTeSR-1/NIM was 3:1 on D1, 1:1 on D2, and complete NIM from D3 to D7. On D7, embryoid bodies were seeded on Matrigel coated 6-well plates and cultured in NIM until D21. On D21, retinal vesicles were picked and cultured in suspension in serum free medium (SFM) comprised of 70% DMEM and 30% F-12 containing 2% B27 and penicillin/streptomycin. For experiments, retinal cups were partially dissociated using 0.2% trypsin (Gibco/Life technologies, Grand Island, N.Y.) and seeded onto GCH-521 (FIG. 12). Medium was changed every 2 to 3 times a week for rest of the experiment.

RNA Extraction

Total RNA extraction was performed using TRIZOL reagent. GCH-521-RPC and GCH-RPC (non-coated; control) was collected in 1.5 ml Eppendorf tubes and centrifuged to remove the culture medium. Trizol (700 μl) was added to the tubes and sonicated for 3 secs in 4° C. Samples were incubated at room temperature for 5 min, and 200 μl of chloroform was added and vortexed vigorously for 10 to 15 seconds. Samples were centrifuges at 12,000×g for 15 min at 4° C. The aqueous phase was removed to fresh tubes without disturbing the interface. Isopropanol was used to precipitate the RNA, and the precipitate was washed with 70% ethanol. The RNA was resuspended in RNase-free water and quantified using a nanodrop spectrophotometer. RNA with A260/A280 ratio 1.8-2.0 was used for qRT²-PCR.

Quantitative Real Time RT-PCR (qRT²-PCR)

RNA with OD between 1.8 to 2.0 was used for cDNA synthesis. cDNA was transcribed using 1 μg of total RNA using i-Script cDNA advance transcription kit (BioRad). Gene tested were for mitotic marker (Ki67), stem cell pluripotency marker (Nanog, Oct-4), early eye field (PAX6, SIX3, LHX2, SIX6, CHX10), retinal progenitor marker (BRN3, Recoverin and NeuroD1) analyzed using i-TaqSYBR green (BioRad) and oligo synthesized (sequence listed in Table 4). Relative mRNA expression was normalized with housekeeping genes like Actin and GAPDH and 2-^{ΔΔCt} method was used for calculation (Livak K J, Schmittgen T D. Methods, 2001, 25:402-408).

TABLE 4
List of primers

| Gene | Cell type | Sequence |
|---|---|---|
| OTX2 | Early eye field | 5′-GCA GAG GTC CTA TCC CAT GA (SEQ |
| | | ID NO: 23) |
| | | 3′-CTG GGT GGA AAG AGA GAA GCTG-5′ |
| | | (SEQ ID NO: 24) |
| NeuroD1 | | |
| GAPDH | Housekeeping gene | 5′-TCA CCA GGG CTG CTT TTA AC-3′ (SEQ |
| | | ID NO: 25) |
| | | 3′-GCA AAG CTT CCC GTT CTC AG-5′ (SEQ |
| | | ID NO: 26) |
| LHX2 | LIM Homeobox | 5′-TAG CAT CTA CTG CAA GGA AGA C-3′ |
| | Protein 2 for neural | (SEQ ID NO: 27) |
| | cells | 3′-GTG ATA AAC CAA GTC CCG AG-5′ |
| | | (SEQ ID NO: 28) |
| NANOG | ES cell | 5′-CAA AGG CAA ACA ACC CAC TT-3′ |
| | proliferation, | (SEQ ID NO: 29) |
| | renewal, and | 3′-TCT GCT GGA GGC TGA GGT AT-5′ (SEQ |
| | pluripotency | ID NO: 30) |
| Oct-4 | Stem cells | 5′-CGA GCA ATT TGC CAA GCT CCT GAA- |
| | pluripotency | 3′ (SEQ ID NO: 31) |
| | marker | 3′-TTC GGG CAC TGC AGG AAC AAA TTC- |
| | | 5′(SEQ ID NO: 32) |
| PAX6 | Neural retinal | 5′-TCT AAT CGA AGG GCC AAA TG-3′ |
| | development | (SEQ ID NO: 33) |
| | | 3′-TGT GAG GGC TGT GTC TGT TC-5′ (SEQ |
| | | ID NO: 34) |
| RAX | Retina and Anterior | 5′-GAA TCT CGA AAT CTC AGC CC-3′ (SEQ |
| | Neural Fold | ID NO: 35) |
| | Homeobox | 3′-CTT CAC TAA TTT GCT CAG GAC-5′ |
| | | (SEQ ID NO: 36) |
| SIX3 | Neural progenitor | 5′-GGA ATG TGA TGT ATG ATA GCC-3′ |
| | cells | (SEQ ID NO: 37) |
| | | 3′-TGA TTT CGG TTT GTT CTG G-5′ (SEQ |
| | | ID NO: 38) |

Immunocytochemistry

Samples were fixed in 4% paraformaldehyde for 5 mins, washed and incubated in graded sucrose solutions up to 30%. Samples were incubated overnight at 4° C. in a 1:1 mixture 30% sucrose and OCT ((Fisher Healthcare™, Pittsburg, Pa.).

Sections, 12 p.m, made with a Leica CM1950 cryostat (Buffalo Grove, IL) at −23° C. and mounted on poly-lysine coated slides. Slides were room dried for 48 hrs before immunocytochemistry. Slides were washed in cold PBS, permeabilized with 0.1% Triton-X100 in PBS (PBST) and blocked with PBST containing 10% donkey serum. The sections were incubated overnight at 4° C. with primary antibodies (Table 5). Next day the slides were washed 3× with PB ST before incubation at room temperature with secondary antibodies conjugated with Cy2, Cy3, or Cy5 (Jackson ImmunoResearch Laboratories, West Grove, Pa.). DAPI (4,6-diamidino-2-phenylindole) was used to label the cell nucleus. Finally, slides were washed 3 more times 3× with PBS. Fluorescence images were captured with an LSM 410 spinning-disc confocal microscope and processed using Zen software (Carl Zeiss, Inc, Thornwood, N.Y.). Images used are representative of 3 or more experiments.

TABLE 5
List of Primary Antibodies

| Target | Antigen | Host1 | Dilution2 | Supplier |
|---|---|---|---|---|
| Anterior | Sox1 | RP | IF 1:400 | Abcam |
| forebrain | | | | |
| Mitotic marker | Ki67 | MP | IF 1:200 | Invitrogen |
| | PAX6 | RM | IF 1:100 | Abgent |
| | | | IB 1:5000 | |
| | RAX | RP | IF 1:200 | Novus Biologicals |
| Early Eye Field | LHX2 | GP | IF 1:300 | Santa Cruz Bio |
| | CHX10 | SP | IB 1:5000 | |
| | OTX2 | MM | IF 1:300 | EMD Millipore |
| | | | IF 1:200 | Novus Biologicals |
| | | | IB 1:3000 | |
| | Recoverin | RP | IF 1:400 | EMD Millipore |
| | | | IB 1:5000 | |
| Photoreceptor | CRX | GP | IF 1:300 | Fisher Scientific |
| Rods/Cones | Rhodopsin | MP | IF 1:300 | Cell Signaling |
| | | | IB 1:3000 | Technology |
| Neural retinal | Prox1 | RM | IF 1:500 | Fisher Scientific |
| cells precursor | β-tubulin III | MP | IF 1:200 | Fisher Scientific |
| Human antigen | TRA-1-85 | MM | IF 1:300 | EMD Millipore |
| | Ku80 | MM | IF 1:250 | Novus |
| Inflammatory | IL-6 | MM | IF 1:200 | Abcam |
| cells | | | | |
| Microglia | IBA-1 | GM | IF 1:200 | Abcam |
| Normalization | Actin | Actin | IB 1:5000 | Sigma |

1RP, Rabbit Polyclonal; RM, Rabbit Monoclonal; MM, Mouse Monoclonal; MP, Mouse Polyclonal; SP, Sheep Polyclonal; GP, Goat Polyclonal
2IF, Immunofluorescence; IB, Immunoblot

Implantation of GCH-WA09-RPC in RD10 Mice

On postnatal day 30 (P30) when the outer nuclear layer was >75% degenerated, (C. Gargini, et al, J. Comp. Neurol. 2007, 500: 222-238) RD-10 mice were anesthetized by intramuscular injection of a mixture of ketamine (100 mg/kg) and xylazine (10 mg/kg). A small scleral hole was made after conjunctival incision and a local retinal detachment was induced by injecting PBS (1 μl) into the sub retinal space. The scleral incision was enlarged to insert cultures (0.5×0.5 cm) into the dorsal quadrant. After transplantation, the mice were transferred into dark room for 1-2 days, and then maintained in regular animal facility. The animals were euthanized at 3 or 6 weeks post-surgery, and the eyes harvested to process the tissues for immunofluorescence.

Transepithelial Electrical Resistance (TER)

TER was measured using endohm electrodes (World Precision Instruments, Sarasota, Fla.). Measurements were made in a modified medium in which the bicarbonate of DMEM was replaced with 20 mM N-2-hydroxyethylpiperazine-N′-2-ethanaesulfonic acid, pH 7.2. The background resistance (10 Q) was subtracted and the measurement reported as Qxcm².

Multifocal Electroretinography (mfERG)

ERGs were recorded using the RetiMap system designed for rodents (Roland Consult Electrophysiological diagnostic systems, Brandenburg, Germany). Mice were anaesthetized using ketamine and xylazine (company). Pupils were dilated using 2.5% tropicamide eye drops (company). A custom-made silver coil electrode of 0.5mm×3 mm Roland Consult) was placed on corneal surface. The reference silver needle electrode was placed on back on neck and ground electrode at tail of the mice. The responses were amplified 100,000× and a 60 Hz band-pass filtered was applied. The signals were digitalized and acquired with 1024-Hz sampling frequency.

Statistical Analysis

All statistical data are presented as the mean±standard error (SE) unless otherwise stated. At least biological repeats were analyzed in triplicate. Comparisons were made using one-way ANOVA and p values<0.05 were considered statistically significant.

### Results

Monoculture of RPC

WA09 cells were differentiated into retinal cups and seeded on the GCH-521 scaffold. After 21 days, the cells had proliferated and populated entire scaffold (FIG. 12). This behavior contrasts with GCH scaffold without laminin, on which cells grew in clusters that failed to coalesce into a continuous sheet. As might be expected from the stem cell preserving properties of laminin 521, expression of the stem cell markers Nanog and Oct-4 increased slightly in retinal cups after plating cells on the scaffold, but decreased rapidly thereafter (FIG. 13A and FIG. 13B). Gene expression of markers for the various retinal cell types was compared for retinal cups seeded on the GCH or GCH-521 scaffold. When expression of mRNA was normalized to a set of housekeeping genes, no appreciable difference was observed on D51 using the broad screen shown in FIG. 5A-FIG. 5C. A few specific examples are shown for possible changes on D31 (FIG. 13C) and D67 (FIG. 13D) At the level of mRNA significant levels of expression were observed for markers of a variety of retinal cell types on D67.

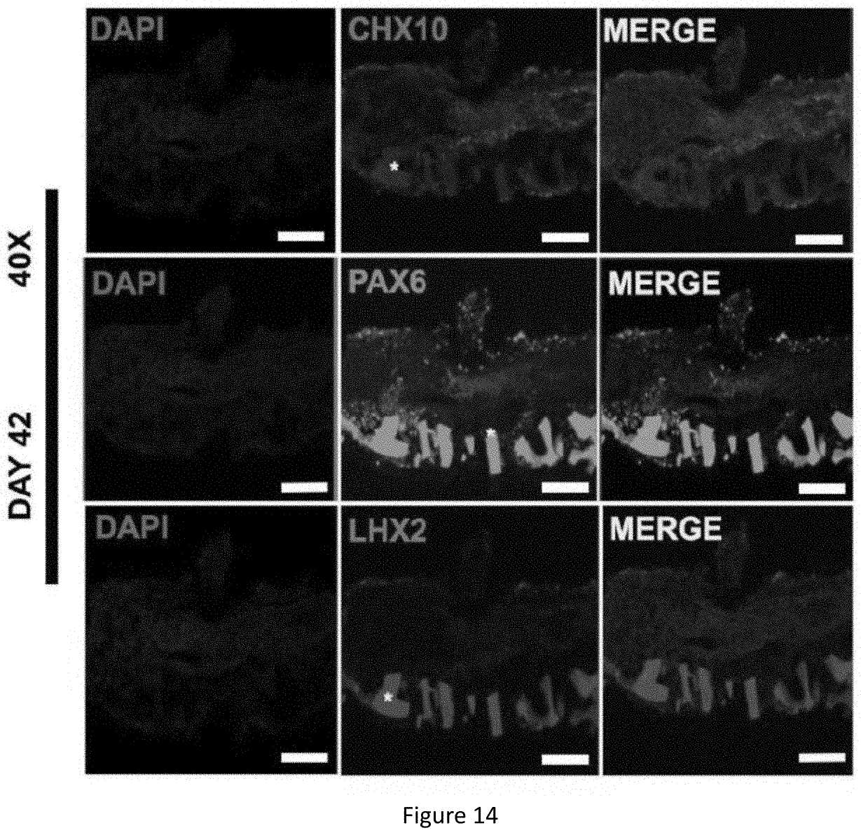

The expression and location of retinal cell types with the neo-tissue was determined by immunofluorescence. Early eye field genes LHX2, CHX10 (VSX2), and PAX6 were evident of D42 (21 days post-seeding). The cells were widely distributed within the scaffold and the thick layer of cells found on top of it (FIG. 14).

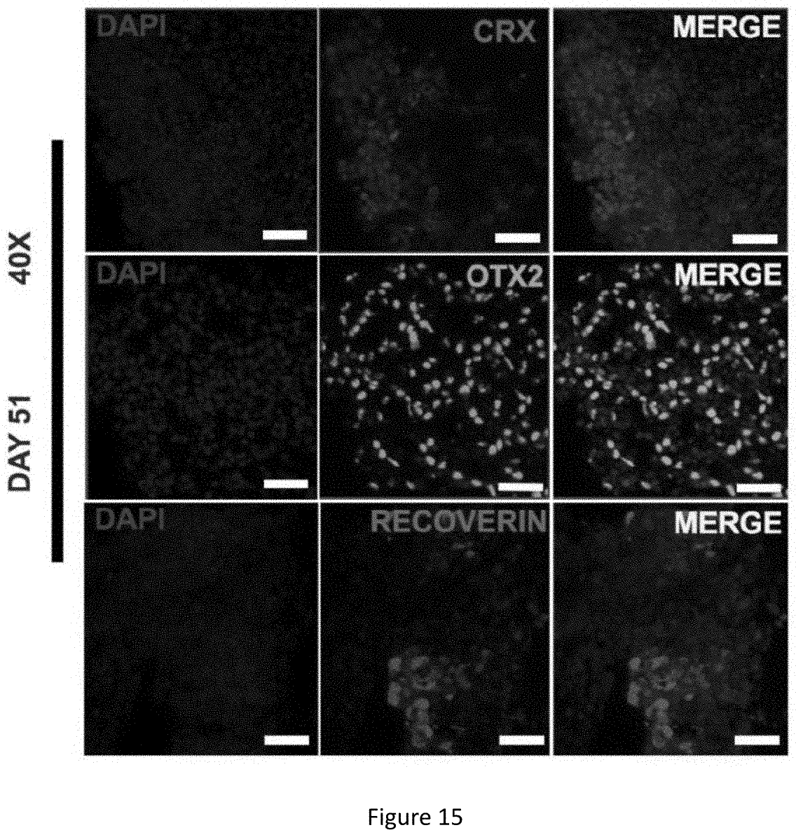

At D51, immature photoreceptor immune staining of CRX, OTX2 and Recoverin expression was observed (FIG. 15). Crx is cone-rod homobox gene which is expressed in both photoreceptor and pinealocytes. Expression of Crx increases committed photoreceptor precursors and key regulator of photoreceptor specific genes. OTX2 plays pivotal role in photoreceptor cell fate determination along with bipolar cell development. It is found upstream of Crx and at later stage together with Crx is involved in both photoreceptor and bipolar cell maturation. Recoverin is neuronal calcium binding protein that is mainly detected in photoreceptor cells. It plays key role in rhodopsin inhibition which in turn regulates sensory adaptation of retina. Recoverin positive cells were initially found around the outer surface of scaffold however, majority of these cells were localized away from the scaffold, near the free surface.

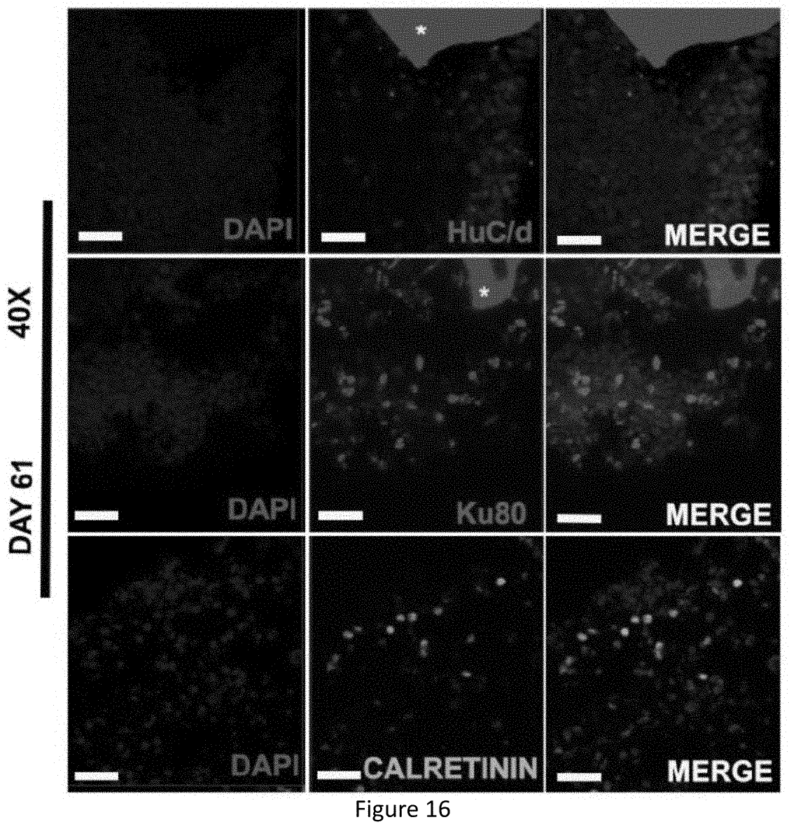

D61, HuC/D and Calretinin marker for neuronal and amacrine cells respectively (FIG. 16). Calretinin is a marker for rod pathway interneuron or AII amacrine cells in the retina. It plays crucial role in nighttime vision and mostly found in the inner nuclear layer of the retina. HuC/D is neuronal cell marker and in retina is used for identifying retinal ganglion cells. In the development stage, it is also found in the horizontal cells but not in the mature types. Expression of mature markers such as Huc/D and calretinin at D61 is clear indication of ability of scaffold to support RPC differentiation in long term culture. Ku protein is involved in my cellular function such as DNA replication, cell cycle regulation and transcriptional activation and often used for identifying human cell population. Presence of calretinin and HuC/D positive cells on GCH-521 indicates scaffold support towards photoreceptor differentiation from hRPC.

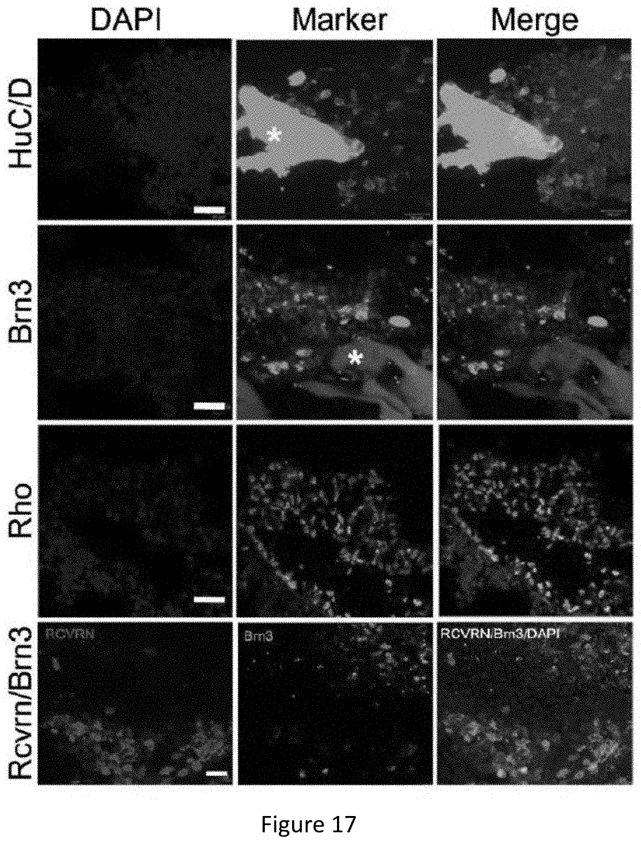

On D77, HuC/D was still expressed along with Brn3 for ganglion cells and rhodopsin for rod photoreceptor marker (FIG. 17). Rhodopsin was towards the outer side of the scaffold where as Brn3 and HuC/D was found towards the scaffold side these results along with D61 indicates self-lamination or segregation of cells within the scaffold was possibly occurring.

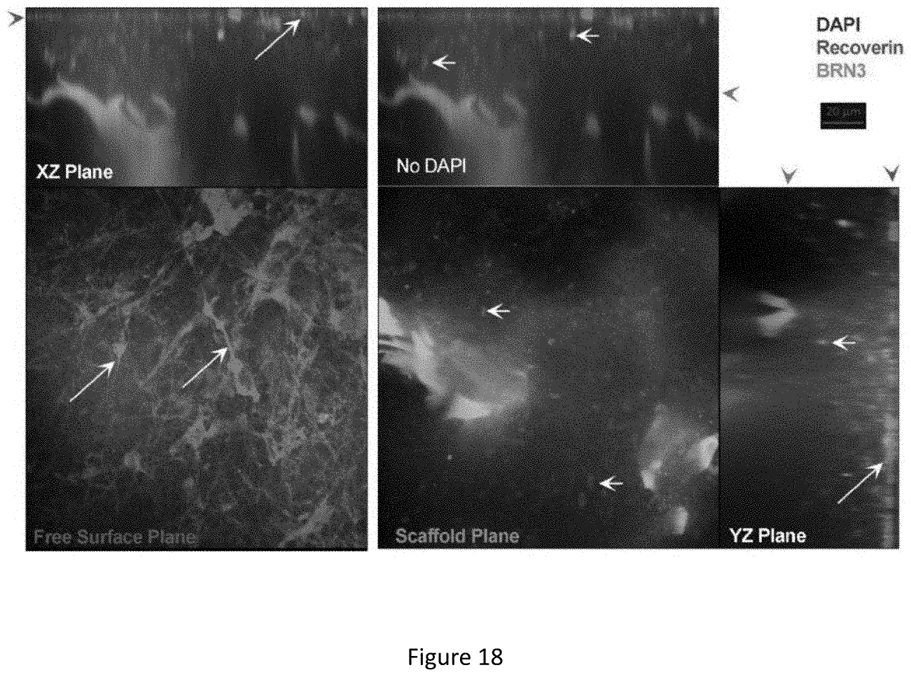

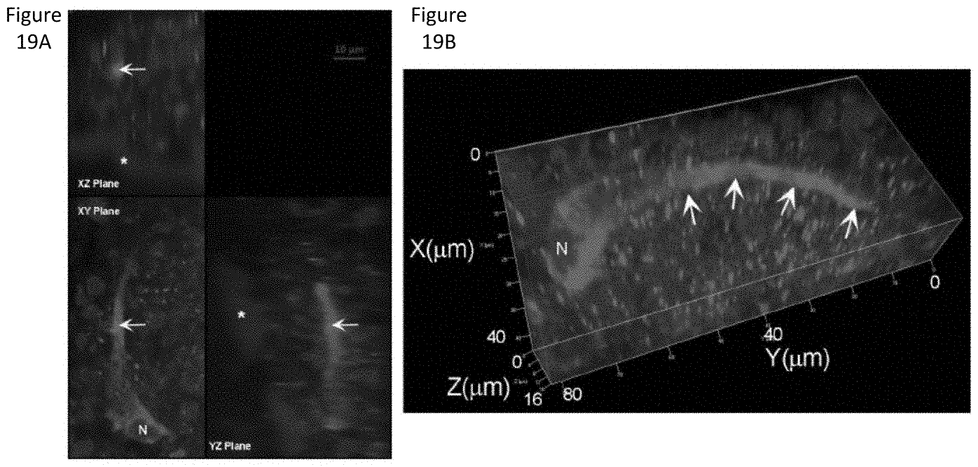

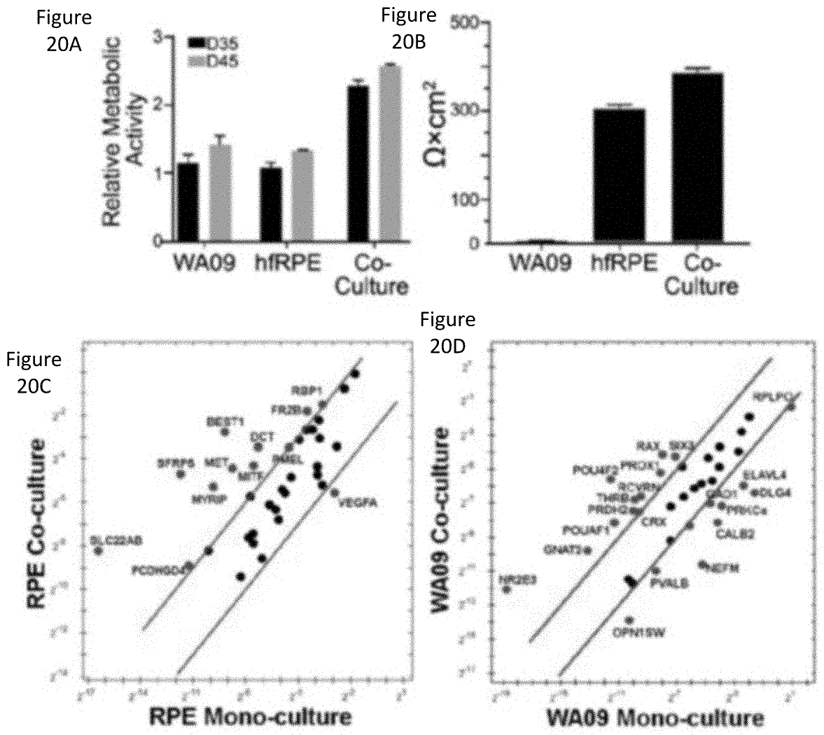

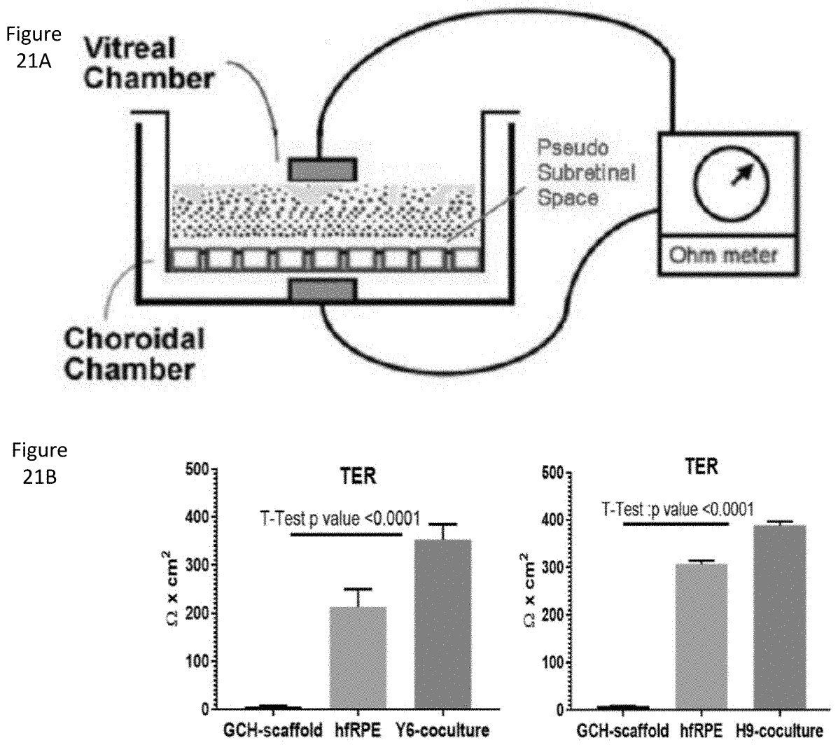

Cultures were maintained up to 8 months. Brn3-positive cells were observed near the scaffold, demonstrating that retinal ganglion cells are long-lived in this culture model. By contrast, ganglion cells die ˜D90-100 in spherical retinoids. Many recoverin-positive cells with long thin and thick cellular extensions were observed near the free surface (FIG. 18). Surprisingly, rhodopsin-positive outer segments were not observed. Cone-shaped cells were evident. Processes as long as 70 μm were identified by antibodies to red-green opsin (FIG. 19A-FIG. 19B). These were found towards the free surface of the culture, away from the scaffold. Although sharp boundaries were not evident between retinal layers, retinal cells did segregate into peri-scaffold, and peri-free surface populations reminiscent of retinal lamina. Interneurons were not evident.

Co-Culture of RPC with RPE

On day 25, RPC were plated on the GCH scaffold and 3 days later were co-cultured with human fetal RPE (hfRPE). The hfRPE had been >6 weeks post confluence and adapted to serum free medium when the scaffold was added to the hfRPE culture. The cultures were followed for up to another 62 days (D90). The metabolic activity, as measured by the Alamar Blue assay, of the co-culture equaled the combined activity of RPE alone plus RPC alone. Therefore, all of the cells appeared to be metabolically active. (FIG. 20A). The TER was measured as illustrated in FIG. 21A-FIG. 21B. The TER of the RPE increased significantly (p<0.01), as a result of coculture for both human embryonic stem cell- and human induced pluripotent stem-derived tissues (FIG. 20B and FIG. 21B).

Gene expression of a collection signature genes for RPE increased because of co-culture, along with two genes that are makers for RPE maturation (SLC22AB and PCDHGD4). Gene expression also increased for many photoreceptor and retinal ganglion cell markers. Gene expression decrease for interneuron markers. Conclusions for Muller cells were ambiguous with expression for some markers increased, while others decreased (FIG. 20C and FIG. 20D).

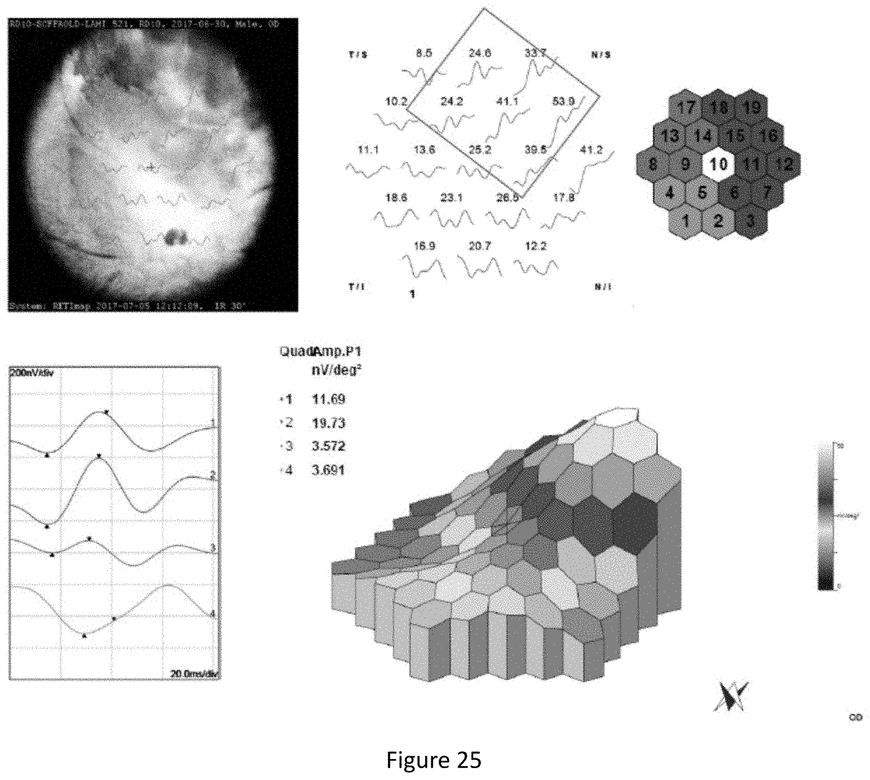

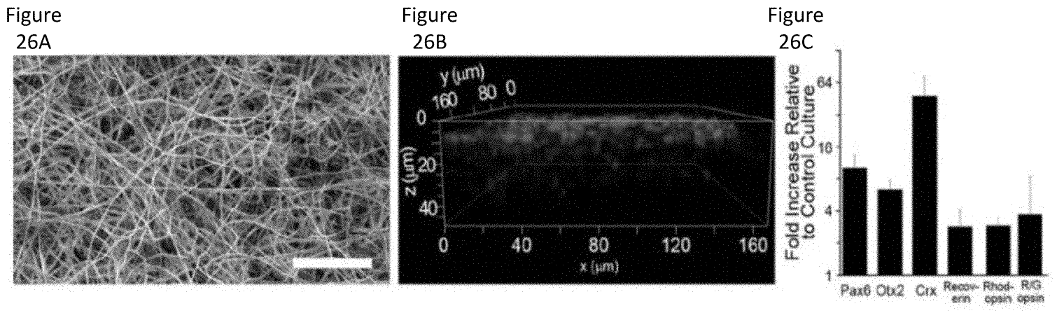

The gene expression data was consistent with the immunocytochemical analysis (FIG. 22A-FIG. 22C). RPE provided a polarity cue to create lamina in the absence of laminin-521, and a thick layer of photoreceptor precursors. Control mono-cultures expressed different cell types, but there was little polarity. An early eye field marker that is also a Muller cell marker, LHX2, co-localized with the photoreceptor precursors and in a layer of cells adjacent to them. This second layer is where one would expect to find interneurons and Muller cells. LHX2, was sparse in and about the scaffold, where retinal ganglion cell precursors are found.

Sub-Retinal Implantation of Immature RPC

The GCH-521-hRPC scaffold was implanted to check for the safety and integration of these grafts in the subretinal space. Sub-retinal transplantation performed in P30 RD10 mice and monitored for 3 weeks to 10 weeks. Immunocytochemical analysis indicated scaffold was successfully placed in the subretinal space (FIG. 23A). The size of the graft was subsequently reduced to reduce the damage to host retina. However, in the higher magnification TRA-1-85 (human antigen) positive cells were found around the scaffold and few migrated into the different layers of the retina (FIG. 23B). TRA-1-85 cells extended its processes into the inner nuclear layer and inner plexiform layer of the mice retina. Interestingly, not only did the implanted cells move out of the scaffold, host cells were found to migrate into the scaffold too. As seen in the FIG. 23C recoverin positive cells in and around the scaffold suggest the host and graft interaction and migration of the cells. To determine if the transplanted cells integrated within the retinal circuitry of the host retina immunocytochemical analysis was performed. There was clear co-localization of PKC-α which is the bipolar cells marker and human antigen marker Ku80. As seen in FIG. 24, colocalization of these markers with Ku80 positive cells extending its processes into INL and IPL layer. The DAPI staining is performed to maintain the sense of retinal orientation and it could be seen few DAPI cells were perfectly lined up with the processes of Ku80 positive cells.

To determine if the integration resulted in the functional recovery of the retina mfERG testing was performed. At 10 weeks post-transplantation (P120), the host retinal function is undetected due to the extensive degeneration of the photoreceptor cells. The non-implanted eyes were treated as control and in the eye with graft showed ˜20 82 V increase in the P1-wave amplitude (FIG. 25). The scaffold could be seen using the fundus image and was used for tracking the implanted area. Within the implanted eye there was clear marked difference in the P1-wave amplitude in the quadrant with the graft versus quadrants without the graft. The difference in the recovered P1-wave amplitude could be due to many factors including addition of new photoreceptor or formation of new connections between new implanted cells with host retina.

### Example 3

### Culture on Electrospun PCL Yields an Incomplete Planar Retinal Organoid

Retinal progenitor cells, were cultured on electrospun PCL, as described for the GCH scaffolds in the previous two examples, and co-cultured with retinal pigment epithelium, as described in Example 2. As demonstrated in FIG. 24, the retinal culture failed to fully populate the scaffold. Nonetheless, co-culture did affect gene expression in retinal progenitors.

### Example 4

### The Culture Model Can Be Used to Test Putative Pharmaceutical Agents

Retinal progenitor cells, were cultured on GCH-laminin 521, as described in Example 2. As demonstrated in FIG. 27, the addition of BDNF to the vitreal medium chamber (FIG. 21A) promoted the maturation of retinal ganglion cells.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A scaffold for culturing retinal tissue comprising:
laminin,
a three-dimensional monolith, wherein the monolith is sectioned into planar sheets, and
wherein the monolith comprises gelatin, chondroitin sulfate, and hyaluronic acid.

2. The scaffold of claim 1, wherein the laminin is laminin-521.

3. The scaffold of claim 1, wherein the three-dimensional monolith is formed by crosslinking.

4. The scaffold of claim 1, wherein the three-dimensional monolith is frozen and lyophilized.

5. The scaffold of claim 1, wherein the scaffold is seeded with cells, wherein the cells are retinal progenitor cells.

6. The scaffold of claim 5, wherein the retinal progenitor cells are derived from human embryonic stem cells.

7. The scaffold of claim 5, wherein the retinal progenitor cells are derived from human inducible pluripotent stem cells.

8. The scaffold of claim 1, wherein the scaffold is seeded on top of a monolayer of cells, wherein the monolayer of cells are retinal pigment epithelial cells.

9. The scaffold of claim 8, wherein the retinal pigment epithelial cells are human fetal retinal pigment epithelial cells.

10. The scaffold of claim 8, wherein the retinal pigment epithelial cells are derived from stem cells, wherein the stem cells are selected from the group consisting of human embryonic stem cells and human inducible pluripotent stem cells.

11. The scaffold of claim 1, wherein the monolith comprises a ratio of concentrations of gelatin, chondroitin sulfate, and hyaluronic acid, wherein the ratio is 2:1:2.

12. The scaffold of claim 1, wherein the planar sheets comprise a thickness of about 60 μm.

13. A retinal coculture system for generating retinal implants comprising:
a planar scaffold comprising an amount of gelatin, chondroitin sulfate, and hyaluronic acid;
a monolayer of differentiated retinal pigment epithelial cells; and
a population of retinal progenitor cells;
wherein the planar scaffold is seeded with cells from the population of retinal progenitor cells, is placed on top of the monolayer of differentiated retinal pigment epithelial cells and is incubated with media.

14. The retinal coculture system of claim 13, wherein the planar scaffold further comprises laminin-521.

15. The retinal coculture system of claim 13, wherein the retinal pigment epithelial cells are human fetal retinal pigment epithelial cells.

16. The retinal coculture system of claim 13, wherein the retinal progenitor cells are derived from human embryonic stem cells.

17. The retinal coculture system of claim 13, wherein the retinal progenitor cells are derived from human inducible pluripotent stem cells.

18. A method of generating retinal implants, the method comprising:
a) generating a scaffold for culturing retinal tissue comprising an amount of gelatin, an amount of chondroitin sulfate, an amount of hyaluronic acid, wherein the amount of gelatin, chondroitin sulfate, and hyaluronic acid are prepared into a three-dimensional monolith, wherein the monolith is sectioned into planar sheets, and an amount of laminin-521;
b) seeding the scaffold with retinal progenitor cells;
c) placing the seeded scaffold in direct contact with a monolayer of retinal pigment epithelial cells; thereby creating a coculture assembly;
d) incubating the coculture assembly thereby generating an organoid; and
e) implanting the generated organoid into the subretinal space of a subject.

19. The method of claim 18, wherein the retinal progenitor cells are human embryonic stem cells.

20. The method of claim 18, wherein the retinal pigment epithelial cells are human fetal retinal pigment epithelial cells.
